(19) United States
(12) Patent Application Publication
(10) Pub. No.: US 2021/0062131 A1
(43) Pub. Date: Mar. 4, 2021

(54) In Vitro, Multi-Niche, Bone Marrow-on-a-Chip

(71) Applicant: Georgia Tech Research Corporation, Atlanta, GA (US)

(72) Inventors: Krishnendu Roy, Atlanta, GA (US); Michael Nelson, Atlanta, GA (US)

(21) Appl. No.: 17/046,163

(22) PCT Filed: Apr. 11, 2019
(86) PCT No.: PCT/US19/26892

§ 371 Date: Oct. 8, 2020

Related U.S. Application Data
(60) Provisional application No. 62/656,040, filed on Apr. 11, 2018.

(51) Int. Cl. C12M 3/06; C12M 1/00; G01N 33/50

(52) U.S. Cl. CPC C12M 23/16; G01N 2800/7028; G01N 33/502; C12M 23/20

(57) ABSTRACT

A multichannel multifluidic device is disclosed that can be configured to mimic the distinct microenvironments of bone marrow by providing different cell types within each channel and culturing the device under conditions to promote development of the distinct mircorenvironments.

### CROSS-REFERENCE TO RELATED APPLICATIONS

This application claims priority to U.S. Provisional Application No. 62/656,040, filed on 11 Apr. 2018, the disclosure of which is herein incorporated by reference in its entirety.

### GOVERNMENT SPONSORSHIP

This invention was made with government support under Grant Nos. DGE-1650044 and DGE-0965945, each awarded by the National Science Foundation. The government has certain rights in the invention.

### BACKGROUND OF THE DISCLOSURE

### 1. Field of the Disclosure

Embodiments of the present disclosure relates generally to small-scale, in vitro microfluidic bone marrow-on-chips, and more specifically to bone marrow-on-chips that mimic the multiple physiological environments present in bone marrow, particularly human bone marrow.

### 2. Background

Human hematopoietic stem cells (hHSCs) and hematopoietic progenitor cells occupy distinct niches within the bone marrow (BM) and are instructed to maintain multipotency through senescence, to proliferate, or to differentiate by signals in the occupied niche. The BM can be divided into three microenvironments: the endosteal niche, the central marrow, and the perivascular niche. HSCs are thought to occupy a perivascular niche, supported by endothelial and perivascular stromal cells, while progenitor cells and hematopoietic differentiation is occurring near the endosteal surface. HSC migration in to and out of the BM is important clinically during BM transplant (BMT); specifically, for the mobilization of HSCs before and the engraftment of HSCs after radiation or chemotherapy. Understanding the cellular and biochemical control over HSC migration is important for improving these clinical procedures and their outcomes on patients. Currently, the BM microenvironment is studied in animal models and by imaging protocols. There is no platform that recreates the complex human BM microenvironments in a defined and tunable system.

What is needed, therefore, is a system that consistently and correctly recreates the distinct niches of human BM in a platform that can be easily manipulated. The system should take advantage of microfluidic devices and cell culture techniques to produce a microfluidic BM-on-a-chip device (BMOC) that accurately mimics the distinct physiological environments present in BM including the different cell types within each environment. The system and microfluidic BMOC device should provide improved methods of studying human BM in a clinical and/or diagnostic setting. It is to such a system and device that embodiments of the present disclosure are directed.

### BRIEF SUMMARY OF THE DISCLOSURE

As specified in the Background Section, there is a great need in the art to identify technologies for improving models of BM, particularly human BM, and use this understanding to develop novel systems and in vitro or microfluidic devices that accurately mimic the distinct niches and cell types found in BM. The present disclosure satisfies this and other needs. Embodiments of the present disclosure relate generally to small-scale, in vitro microfluidic BMOC devices, and more specifically to BMOC devices that mimic the multiple physiological environments present in bone marrow, particularly human bone marrow.

In one aspect, the present disclosure provides a multichannel multifluidic bone marrow-on-a-chip (BMOC) device comprising: at least three channels; at least one media port; and at least one gel port, wherein the at least three channels comprise at least one central channel and at least two external channels, wherein one external channel is located on one side of the at least one central channel and proximal to the at least one central channel, wherein another external channel is located on the opposite side of the at least one central channel and proximal to the at least one central channel, wherein the at least one central channel comprises: a coating or surface treatment comprising one or more of polydopamine, fibrin, fibrinogen, fibronectin, collagen, hyaluronic acid, extracellular matrix (ECM) proteins or peptides, ECM-like proteins or peptides, PEG and functionalized PEG; and either (i) differentiated cells selected from the group consisting of osteoblasts and osteoclasts; or (ii) undifferentiated cells selected from the group consisting of MSCs, HSCs, HUVECs, pre-OBs, and other MSC-derived bone-lineage cells, wherein the at least one central channel contains one or more of (i) pro-vascularization media, (ii) a pro-vascularization gel suspension comprising one or more of fibrin, fibrinogen, fibronectin, collagen, gelatin, hyaluronic acid, polyethylene glycol, functionalized polyethylene glycol, and combinations thereof, iii) whole tissue comprising bone marrow aspirate and peripheral blood, and (iv) cells selected from the group consisting of endothelial cells, hematopoietic cells, and stromal cells, and wherein, prior to addition of the one or more of (i) pro-vascularization media, (ii) a pro-vascularization gel suspension comprising one or more of fibrin, fibrinogen, fibronectin, collagen, gelatin, hyaluronic acid, polyethylene glycol, functionalized polyethylene glycol, and combinations thereof, iii) whole tissue comprising bone marrow aspirate and peripheral blood, and (iv) cells selected from the group consisting of endothelial cells, hematopoietic cells, and stromal cells, the BMOC device is cultured for a period of about 7 to about 21 days to promote formation of the endosteal surface microenvironment of bone marrow, wherein, after addition of the one or more of (i) pro-vascularization media, (ii) a pro-vascularization gel suspension comprising one or more of fibrin, fibrinogen, fibronectin, collagen, gelatin, hyaluronic acid, polyethylene glycol, functionalized polyethylene glycol, and combinations thereof, iii) whole tissue comprising bone marrow aspirate and peripheral blood, and (iv) cells selected from the group consisting of endothelial cells, hematopoietic cells, and stromal cells, the BMOC device is cultured for a period of about 3 to about 7 days to promote vascularization of the BMOC device and formation of one or both of central marrow and/or periosteal niche microenvironments of bone marrow.

In another aspect, the present disclosure provides a method of producing a bone-marrow-on-a-chip (BMOC) device, the method comprising:

forming a multichannel microfluidic device from PDMS by soft lithography, wherein the BMOC device comprises at least three channels, at least one media port, and at least one gel port,

wherein the at least three channels comprise at least one central channel and at least two external channels,

wherein one external channel is located on one side of the at least one central channel and proximal to the at least one central channel,

wherein another external channel is located on the opposite side of the at least one central channel and proximal to the at least one central channel;

introducing cells, media and adhesion-promoting materials into the at least one central channel to form an endosteal surface microenvironment,
wherein the cells comprise either (i) differentiated cells selected from the group consisting of osteoblasts and osteoclasts; or (ii) undifferentiated cells selected from the group consisting of MSCs, HSCs, HUVECs, pre-OBs, and other MSC-derived bone-lineage cells,
wherein the media comprises commercially available differentiation media or alphaMEM media optionally supplemented with one or more cytokines, differentiation factors, growth factors, fetal bovine serum, dexamethasone, ascorbic acid, beta-glycerophosphate and/or other osteogenic chemicals, cytokines, or additives,
wherein the adhesion-promoting materials comprise collagen, polydopamine, fibrin, fibrinogen, fibronectin, gelatin, polygelatin, ECM proteins or peptides, ECM-like proteins or peptides, silanes, polyethylene glycol, functionalized polyethylene glycol, and combinations thereof, and
wherein the adhesion-promoting materials form a coating in the at least one central channel;

culturing the BMOC device for about 7 days to about 21 days to promote formation of the endosteal surface;

introducing one or more of pro-vascularization cells or tissue, pro-vascularization media, and a pro-vascularization gel into the one or more central channel(s) of the BMOC device to promote vascularization of the BMOC device,
wherein the pro-vascularization cells or tissue comprise endothelial cells, stromal cells, hematopoietic cells, whole tissue, bone marrow aspirate, and peripheral blood,
wherein the pro-vascularization media comprises endothelial growth media optionally supplemented with one or more cytokines, differentiation factors, and growth factors,
wherein the pro-vascularization gel comprises one or more of fibrin, fibrinogen, fibronectin, collagen, gelatin, hyaluronic acid, polyethylene glycol, functionalized polyethylene glycol, and combinations thereof;

introducing pro-vasculogenesis supporting cells into the outer channel(s),
wherein the outer channel(s) are coated with adhesion-promoting materials comprising one or more of collagen, polydopamine, fibrin, fibrinogen, fibronectin, gelatin, polygelatin, ECM proteins or peptides, ECM-like proteins or peptides, silanes, polyethylene glycol, functionalized polyethylene glycol and combinations thereof, and
wherein the pro-vasculogenesis supporting cells comprise MSCs, fibroblasts, or other pro-vasculogenic primary cells or cell lines; and

culturing the BMOC device in vasculogenic media for about 3 to about 7 days to promote vascularization of the BMOC device and formation of one or both of the central marrow and/or periosteal niche microenvironments of the bone marrow.

In another aspect, the present disclosure provides a method of determining the effects of a therapeutic drug on bone marrow comprising: generating a bone marrow-on-chip (BMOC) device as described in any embodiment herein; providing the therapeutic drug to the BMOC device; and assaying the effects of the therapeutic drug on the bone marrow tissue contained in the BMOC device, wherein the assayed effects comprise one or more of toxicity, cell differentiation, stem cell migration/mobilization, cytokine expression, and health of the bone marrow tissue or cells, and wherein the assay comprises one or more of measuring cytokine levels, detecting specific cytokines, microscopy, immunohistochemistry, cell tracking and imaging, vascular network analysis and imaging, cell engraftment analysis, measurement of cell death or killing, and recovery of cells or gels from the BMOC device for single or multi-cell analysis comprising RNA-seq, flow cytometry, Western blotting, ELISA, quantitative PCR, and nucleic acid hybridization and detection.

In a related aspect, the present disclosure provides a method of characterizing a hematopoietic malignancy or other metastatic malignancy comprising: generating a bone marrow-on-chip (BMOC) device as described in any embodiment herein, wherein the cells introduced into the at least one channel comprise cells from known malignant cell lines or cells from a sample from a patient having a hematopoietic malignancy; and assaying the effects of the hematopoietic malignancy or other metastatic malignancy on the bone marrow tissue contained in the BMOC device, wherein the assayed effects comprise one or more of toxicity, cell differentiation, stem cell migration/mobilization, cytokine expression, and health of the bone marrow tissue or cells, and wherein the assay comprises one or more of measuring cytokine levels, detecting specific cytokines, microscopy, immunohistochemistry, cell tracking and imaging, vascular network analysis and imaging, cell engraftment analysis, measurement of cell death or killing, and recovery of cells or gels from the BMOC device for single or multi-cell analysis comprising RNA-seq, flow cytometry, Western blotting, ELISA, quantitative PCR, and nucleic acid hybridization and detection.

In a related aspect, the present disclosure provides a method of measuring bone marrow stem cell mobilization comprising: generating a bone marrow-on-chip (BMOC) device as described in any embodiment herein, wherein the cells introduced into the at least one channel comprise bone marrow stem cells from known cell lines or bone marrow stem cells collected by leukophoresis from a patient; and assaying the mobility of the bone marrow stem cells contained in the BMOC device, wherein the assay comprises one or more of measuring cytokine levels, detecting specific cytokines, microscopy, immunohistochemistry, cell tracking and imaging, vascular network analysis and imaging, cell engraftment analysis, measurement of cell death or killing, and recovery of cells or gels from the BMOC device for single or multi-cell analysis comprising RNA-seq, flow cytometry, Western blotting, ELISA, quantitative PCR, and nucleic acid hybridization and detection.

In a related aspect, the present disclosure provides a method of characterizing the effects of radiation exposure on bone marrow comprising: generating a bone marrow-on-chip (BMOC) device as described in any embodiment herein, wherein the cells introduced into the at least one channel comprise cells from known cell lines or cells from a sample from a patient; and assaying the effects of the radiation exposure on the bone marrow tissue contained in the BMOC device, wherein the assayed effects comprise one or more of toxicity, cell differentiation, stem cell migration/mobilization, cytokine expression, and health of the bone marrow tissue or cells, and wherein the assay comprises one or more of measuring cytokine levels, detecting specific cytokines, microscopy, immunohistochemistry, cell tracking and imaging, vascular network analysis and imaging, cell engraftment analysis, measurement of cell death or killing, and recovery of cells or gels from the BMOC device for single or multi-cell analysis comprising RNA-seq, flow cytometry, Western blotting, ELISA, quantitative PCR, and nucleic acid hybridization and detection.

These and other objects, features and advantages of the present disclosure will become more apparent upon reading the following specification in conjunction with the accompanying description, claims and drawings.

### BRIEF DESCRIPTION OF THE DRAWINGS

The accompanying Figures, which are incorporated in and constitute a part of this specification, illustrate several aspects described below.

FIG. 1 depicts an exemplary three channel BMOC device according to the present disclosure.

FIG. 2 depicts an iteration of an exemplary five channel BMOC device.

FIG. 3 depicts another iteration of an exemplary five channel BMOC device.

FIGS. 4A-4B shows an exemplary five channel BMOC device (FIG. 4A) and an exploded cross section of the device showing a time course of cell development within the individual channels of a five channel BMOC device (FIG. 4B).

FIGS. 5A-5C show a diagram of an exemplary five channel BMOC device (FIG. 5A), pores that connect the channels of the device (FIG. 5B), and a diagram showing the relationship of the channels to one another (FIG. 5C).

FIGS. 6A-6C show the three different fabrication processes for the BMOC devices shown in FIGS. 2-3.

FIGS. 7A-7B show that osteogenic differentiation of hMSCs resulted in substantial deposition of mineralized matrix after 21 days of differentiation by staining with Alizarin (FIG. 7A) and Kossa (FIG. 7B).

FIG. 8 shows vasculogenesis of a BMOC device provided with a fibrin gel comprising pro-vascularization factors VEGF and adiponectin-1.

FIG. 9 shows vasculogenesis of a BMOC device provided with a fibrin and collagen gel comprising pro-vascularization factors VEGF and adiponectin-1. Differentiation of OBs began on day −21, HUVECs (with or without MSCs) were added on day 0, VEGF was added on day 1, VEGF and adiponectin-1 were added on days 2-4, and the cells in the device were fixed and stained on day 5. It was found that OB containing BMOC devices had longer vascular networks but the same % area, suggesting that although average vessel diameter is less, however the difference is not significant. n=6, mean±SD.

FIG. 10 shows secretion of cytokines into the supernatant from the BMOC device shown in FIG. 9 on day 5. Higher levels of hematopoietic cytokines were detected in devices with OBs than without OBs. n=4, mean±SEM, Kruskal-Wallis w/Dunn's Multiple Comparisons Test.

FIG. 11 shows secretion of cytokines from the BMOC device shown in FIG. 9, where the cytokines were harvested from the supernatant and the gel. A higher level of cytokines were present in the supernatant relative to the hydrogel. S=Supernatant, H=Hydrogel; n=4, mean±SEM, Two-Way ANOVA w/Sidak's Multiple Comparisons Test.

FIG. 12 shows the application of culturing BM derived hematopoietic stem and progenitor cells (HSPCs) in BMOC. BM HSPCs expanded more in devices without an endosteal layer indicating that there is a difference in function depending on the inclusion of different stromal cells. (Top panel) Raw number of HSPCs (left individual data, righ—summary data). (Bottom panel) Fold change number of HSPCs on days 1, 3, and 5 (left individual data, right summary data). Data (n=4 devices EC+OB, n=7 devices EC+MSC +OB, n=8 devices EC and EC+MSC) are shown as mean±SEM.

FIG. 13 shows secretion of cytokines from a BMOC device provided with a fibrin and collagen gel comprising pro-vascularization factors VEGF and adiponectin-1. Differentiation of OBs began on day −21, HUVECs and human pluripotent stem cells (HPSCs), with or without MSCs were added on day 0, VEGF was added on day 1, VEGF and adiponectin-1 were added on days 2-4, and the supernatant and hydrogel from the device were collected on day 5. n=6, mean±SEM, Kruskal-Wallis w/Dunn's Multiple Comparisons Test.

FIG. 14 shows cytokine secretion from a BMOC device without pro-vascularization factors. n=4-6, mean±SD.

FIG. 15 shows the effects of mobilizing agents G-CSF and AMD3100 on bone marrow microenvironments measured by cytokine expression in a BMOC device where OBs began differentiation on day −26, HUVECs and MSCs were added on day −5, VEGF was added on day −4, VEGF and adiponectin-1 were added on day −3, and G-CSF and AMD3100 were added at hour 0. Samples of supernatant from the BMOC device were collected before the addition of G-CSF and AMD3100 and 24 hours after the addition of G-CSF and AMD3100. The change in level of each cytokine was determined by subtracting the amount of the cytokine before adding the mobilizing agent from the amount of cytokine 24 hours after adding the mobilizing agent. n=5, mean±SEM, Kruskal-Wallis w/Dunn's Multiple Comparisons Test.

FIG. 16 shows that G-CSF can decrease expression of cytokine CXCL12 in a BMOC where OBs began differentiation on day −21, HUVECs and MSCs were added on day 0, and G-CSF and AMD3100 were added on day 5. Cells were fixed and stained on day 6. CXCL12 expression was measured by immunohistochemistry (IHC); n=2-3 devices (× 5 ROI each), mean±SD.

FIG. 17A-17D show the effects of radiation (time course in FIG. 17A) on cytokine secretion (FIG. 17B) from a BMOC device where OBs began differentiation on day −26, HUVECs and MSCs were added on day −5, VEGF was added on day −4, VEGF and adiponectin-1 were added on day −3, and X-ray exposure began at hour 0. Media were collected and stained at hour 24. The change in level of each cytokine was determined by subtracting the amount of the cytokine before X-ray exposure from the amount of cytokine 24 hours after X-ray exposure. n=4, mean±SEM, Kruskal-Wallis w/Dunn's Multiple Comparisons Test. X-ray radiation exposure resulted in no measurable cytoxicity (FIG. 17C), but an increased expression of cytokine CXCL12 (FIG. 17D).

FIG. 18 shows an application of a microfluidic device optimized to grow K562 cells that are models for leukemia. It was shown that K562 cells proliferated within vasculature-on-chip and BMOC devices over a 4-day period. The fold change in K562 count at each time point was not significantly different in the presence of an osteoblast (OB) layer (2-way ANOVA, p<0.05).

FIG. 19 shows an application of a microfluidic device optimized to grow metastatic breast tumor cells. MDA-MB-231 (breast adenocarcinoma cell line) cocultured with HUVECs and MSCs were able to proliferate within the microfluidic vasculature-on-chip device.

### DETAILED DESCRIPTION OF THE DISCLOSURE

As specified in the Background Section, there is a great need in the art to identify technologies for improving models of BM, particularly human BM, and use this understanding to develop novel systems and in vitro or microfluidic devices that accurately mimic the distinct niches and cell types found in BM. The present disclosure satisfies this and other needs. Embodiments of the present disclosure relate generally to small-scale, in vitro microfluidic BMOC devices, and more specifically to BMOC devices that mimic the multiple physiological environments present in bone marrow, particularly human bone marrow.

To simplify and clarify explanation, the system is described below as a system for mimicking the distinct niches within bone marrow, particularly human bone marrow.

To facilitate an understanding of the principles and features of the various embodiments of the disclosure, various illustrative embodiments are explained below. Although exemplary embodiments of the disclosure are explained in detail, it is to be understood that other embodiments are contemplated. Accordingly, it is not intended that the disclosure is limited in its scope to the details of construction and arrangement of components set forth in the following description or examples. The disclosure is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present disclosure as many comparable parameters, sizes, ranges, and/or values may be implemented. The terms “first,” “second,” and the like, “primary,” “secondary,” and the like, do not denote any order, quantity, or importance, but rather are used to distinguish one element from another.

It is noted that terms like “specifically,” “preferably,” “typically,” “generally,” and “often” are not utilized herein to limit the scope of the claimed disclosure or to imply that certain features are critical, essential, or even important to the structure or function of the claimed disclosure. Rather, these terms are merely intended to highlight alternative or additional features that may or may not be utilized in a particular embodiment of the present disclosure. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm.”

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present disclosure are intended to be illustrative and not restrictive. Many suitable materials that would perform the same or a similar function as the materials described herein are intended to be embraced within the scope of the disclosure. Such other materials not described herein can include, but are not limited to, materials that are developed after the time of the development of the disclosure, for example. Any dimensions listed in the various drawings are for illustrative purposes only and are not intended to be limiting. Other dimensions and proportions are contemplated and intended to be included within the scope of the disclosure.

As used herein, the term “subject” or “patient” refers to mammals and includes, without limitation, human and veterinary animals. In a preferred embodiment, the subject is human.

A “disease” is a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “therapeutic” as used herein means a treatment and/or prophylaxis. A therapeutic effect is obtained by suppression, diminution, remission, or eradication of a disease state. As used herein the term “therapeutically effective” applied to dose or amount refers to that quantity of a compound or pharmaceutical composition that when administered to a subject for treating (e.g., preventing or ameliorating) a state, disorder or condition, is sufficient to effect such treatment. The “therapeutically effective amount” will vary depending on the compound or bacteria or analogues administered as well as the disease and its severity and the age, weight, physical condition and responsiveness of the mammal to be treated.

In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

In accordance with the present disclosure there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985); Transcription and Translation (B. D. Hames & S. J. Higgins, eds. (1984); Animal Cell Culture (RI. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

### Bone Marrow-on-Chip Devices of the Disclosure

Devices of the present disclosure include multi-channel microfluidic devices that are organized in a way to generate distinct microenvironments found in BM in and across the channels, by using for example and not limitation, different culture media, supplements (e.g., differentiation factors and cytokines), and cell types. The distinct microenvironments created in the channels of the BMOC device enable mimicking of different niches or microenvironments found in bone marrow (BM). In some embodiments, the channels are configured to enable transfer of the contents of one channel, such as for example and not limitation, culture media, differentiation factors, and/or cytokines to another channel, e.g., from an outer channel to an inner channel.

In one aspect of the present disclosure is provided a microfluidic device with at least three channels. The central channel(s) are coated to promote cellular adhesion of the desired cell types. In some embodiments, the channels are coated or treated with different substances to promote adhesion of different cell types in the different channels. For example and not limitation, the central channel(s) can be coated with one substance(s) and the outer channel(s) can be coated with a second substance(s). In some embodiments, the channels can be coated or treated with one or more adhesion-promoting materials such as for example and not limitation, polydopamine, collagen (e.g., type I), fibronectin, fibrin, gelatin, polygelatin, extracellular matrix (ECM) proteins or peptides, ECM-like proteins or peptides, and combinations thereof (e.g., polydopamine and collagen type I). In some embodiments, the channels can have chemically modified surfaces (e.g., a silane such as (3-aminopropyl)triethoxy silane (APTES)) to promote adhesion, alone or in combination with other adhesion-promoting materials. In some embodiments, the channels are coated with artificial adhesion-promoting polymers such as polyethylene glycol (PEG) and modified versions of PEG (e.g., PEG modified with an adhesion-promoting material such as a protein or peptide).

In some embodiments, the device comprises three channels, which include a central gel channel and two outer media channels. In other embodiments, the device comprises five channels, which include a central gel channel, two media channels, and two outer gel channels. In other embodiments, the device comprises seven channels, which include a central gel channel, two media channels, two outer gel channels, and two outer media channels.

In some embodiments, the device comprises hydrophobic surfaces within the media channels and outer gel channels, which enable the formation of air-liquid interfaces between cell and media-containing channels and the adjacent empty media channels. In some embodiments, the hydrophobic surfaces are formed by coating with a hydrophobic substance, such as for example and not limitation, polysiloxanes (e.g., hydroxy-terminated polydimethylsiloxane, octadecyltrichlorosilane). In some embodiments, the material from which the device is made is itself hydrophobic and no coating is needed, such as for example and not limitation, PDMS. Without wishing to be bound by theory, it is suggested that the hydrophobic surfaces deter unwanted wetting of media channels adjacent to the central channel which enables prolonged, isolated culture of cells within the central channel and subsequent isolated loading of additional cells and or hydrogel on top of the cultured cell layer.

In some embodiments, different cell types are introduced into different channels of the BMOC device. In some embodiments, the cell types have been differentiated before they are introduced into the device channel (e.g., osteoblasts, osteoclasts). In some embodiments, the cell types are not differentiated before they are introduced into the central device channel (e.g., MSCs, pre-osteoblasts, HSCs, MSC-derived bone lineage cells, HUVECs), and instead are differentiated or cultured within the channel by providing suitable differentiation media comprising differentiation factors, cytokines, growth factors, and/or additional cells and waiting for differentiation or self-assembly into 3D tissues to occur as discussed hereinbelow. In some embodiments, the additional cells are introduced into the outer channel(s) of the device, while the undifferentiated cells are introduced into the central channel(s) of the device, and the appropriate differentiation factors, growth factors, cytokines, and other necessary substances are enabled to diffuse out of the outer channel(s) into the central channel(s) to induce differentiation and/or self-assembly of the cells into 3D tissues in the central channel(s). Non-limiting examples of the cell types provided to the BMOC device include mesenchymal stromal cells (MSC), MSC-derived bone-lineage cells, hematopoietic stem cells (HSC), osteoblasts (OB), pre-osteoblasts (pre-OBs), osteoclasts (OCs), human umbilical vein endothelial cells (HUVECs), endothelial cells from other tissue origins, or induced pluripotent stem cell (iPSC) derived cell types. In some embodiments, the cells are obtained from a patient tissue sample or bone marrow aspirate to provide devices with cells that are patient-specific. Cells that can aid in differentiation and/or creation of a device that accurately mimics an organ microenvironment include but are not limited to adipoyctes, neutrophils, macrophages, osteoclasts, and other cells in BM microenvironment.

In some embodiments, the BMOC device is designed to mimic the distinct niches or microenvironments found within human BM. In some embodiments, the BMOC device comprises at least two distinct microenvironments that mimic microenvironments found in BM, particularly human BM, including the endosteal niche, the central marrow, and the perivascular niche. In some embodiments, the BMOC device provides the at least two distinct microenvironments in an in vivo-like anatomically correct configuration, e.g., with the central marrow surrounded by the perivascular niche and then the endosteal niche.

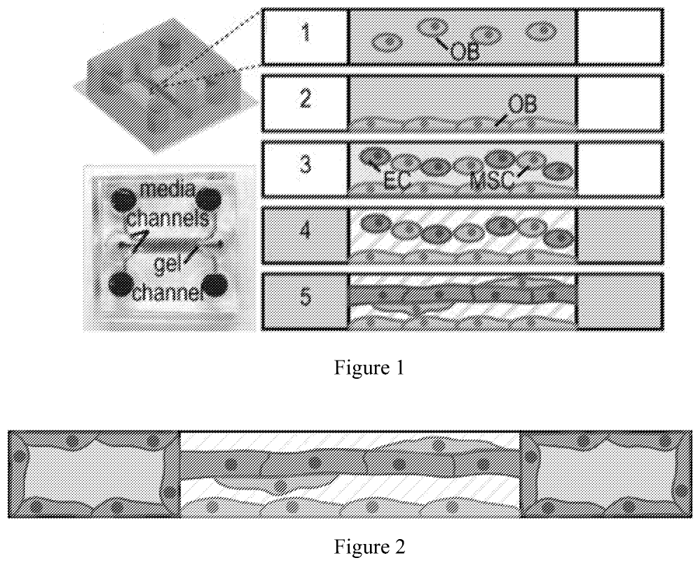

In some embodiments, the BMOC device comprises at least three channels, where the central channels are coated with adhesion-promoting materials and the outer channels are media channels (FIG. 1). In some embodiments, the central gel-coated channel(s) comprise previously differentiated cells such as OBs or OCs to serve as stromal cells in the central marrow and endosteal niche. In other embodiments, the central channel(s) comprise undifferentiated cells such as MSCs, HSCs, HUVECs, pre-OBs, and other MSC-derived bone-lineage cells, and appropriate differentiation media can be subsequently introduced into the central channel(s) to enable differentiation and/or self-assembly into a 3D tissue over a suitable time period. In some embodiments, an air-liquid interface between the central cell-coated channel(s) and the adjacent medial channels is maintained. The differentiation media can comprise, for example and not limitation, commercially available differentiation media (e.g., Stempro Osteo) or alphaMEM supplemented with fetal bovine serum (FBS), dexamethasone, ascorbic acid, beta-glycerophosphate and/or other osteogenic chemicals, cytokines, or additives. In some embodiments, once mineralization and establishment of the endosteal niche has occurred, e.g., within about 7 to about 21 days of differentiation from undifferentiated cells, the existing cell monolayer can be supplemented with a gel containing endothelial cells that is loaded on top of the endosteal surface. In some embodiments, a solution comprising thrombin and fibrinogen can be introduced into the central gel-coated channel(s) to promote formation of a fibrin gel. In some embodiments, the gel comprises natural substances (e.g., fibrin, fibrinogen, thrombin, collagen, hyaluronic acid, and combinations thereof) and/or artificial polymers (e.g., PEG including PEG modified or functionalized with proteins or peptides). In some embodiments, the gel further comprises additional cells and/or tissues to aid in vasculature development, such as (i) endothelial cells (e.g., HUVECs, MVECs, EPCs, other human endothelial cells and combinations thereof), (ii) stromal cells (e.g., MSCs, adipocytes and combinations thereof), (iii) hematopoietic cells (e.g., HSCs, HPCs, HSPCs, macrophages, neutrophils and combinations thereof), and/or (iv) whole tissue (e.g., processed or unprocessed bone marrow aspirate and/or peripheral blood). In some embodiments, endothelial cells, stromal cells, hematopoietic cells and/or whole tissue, optionally in combination with other cell types such as adipocytes, macrophages, and/or other hematopoietic cells, can be suspended in a gel (a nonlimiting example is a fibrin-based gel) and then loaded on top of the “endosteal” surface in the central gel-coated channel(s). In some embodiments, the media channel(s) can be filled with appropriate media (e.g., culture media, differentiation media, and/or pro-vascularization media comprising differentiation factors, cytokines, growth factors and/or additional cell types) such that vasculogenesis can occur. Pro-vascularization media can comprise appropriate commercially available culture media (e.g., endothelial growth media such as EGM-2MV) optionally supplemented with appropriate cytokines and/or differentiation factors, e.g., VEGF and/or Angiopoietin-1. In some embodiments, vasculogenesis occurs over a period of about 3 to about 7 days by culturing in appropriate media, e.g., vasculogenic media. After vasculature has formed, devices are ready to be assayed by various methods described hereinbelow.

In some embodiments, the BMOC device comprises at least five channels, including the central channel(s) coated with adhesion promoting materials, the outer media channels, and additional outer coated channels on the exterior of the media channels (FIG. 2). In some embodiments, the central gel-coated channel(s) comprise previously differentiated cells such as OBs or OCs to serve as stromal cells in the central marrow and endosteal niche. In other embodiments, the central channel(s) comprise undifferentiated cells such as MSCs, HSCs, HUVECs, pre-OBs, and other MSC-derived bone-lineage cells, and appropriate differentiation media can be subsequently introduced into the central channel(s) to enable differentiation and/or self-assembly into a 3D tissue over a suitable time period. In some embodiments, an air-liquid interface between the central gel-coated channel(s) and the adjacent medial channels is maintained. The differentiation media can comprise, for example and not limitation, commercially available differentiation media (e.g., Stempro Osteo) or alphaMEM supplemented with fetal bovine serum (FBS), dexamethasone, ascorbic acid, beta-glycerophosphate and/or other osteogenic chemicals, cytokines, or additives. In some embodiments, once mineralization and establishment of the endosteal niche has occurred, e.g., within about 7 to about 21 days of differentiation from undifferentiated cells, the existing cell monolayer can be supplemented with a gel containing endothelial cells that is loaded on top of the endosteal surface. In some embodiments, a solution of thrombin and fibrinogen can be introduced into the central gel-coated channel(s) to promote formation of a fibrin gel. In some embodiments, the gel comprises natural substances (e.g., fibrin, fibrinogen, thrombin, collagen, hyaluronic acid, and combinations thereof) and/or artificial polymers (e.g., PEG). In some embodiments, the gel further comprises additional cells and/or tissues to aid in vasculature development, such as (i) endothelial cells (e.g., HUVECs, MVECs, EPCs, other human endothelial cells and combinations thereof), (ii) stromal cells (e.g., MSCs, adipocytes and combinations thereof), (iii) hematopoietic cells (e.g., HSCs, HPCs, HSPCs, macrophages, neutrophils and combinations thereof), and/or (iv) whole tissue (e.g., processed or unprocessed bone marrow aspirate and/or peripheral blood). In some embodiments, endothelial cells, stromal cells, hematopoietic cells and/or whole tissue, optionally in combination with other cell types such as adipocytes, macrophages, and/or other hematopoietic cells, can be suspended in a gel (a nonlimiting example is a fibrin-based gel) and then loaded on top of the “endosteal” surface in the central gel-coated channel(s). In some embodiments, MSCs (e.g., from bone marrow or other tissue sources), fibroblasts (e.g., from dermal, lung, or other tissue sources), or other cells from a pro-vasculogenic primary cell or cell line can be suspended in a gel (a nonlimiting example is a fibrin-based gel) and loaded into the outer gel-coated channel(s). In some embodiments, the media channel(s) can be filled with appropriate media (e.g., culture media, differentiation media, and/or pro-vascularization media comprising differentiation factors, cytokines, growth factors and/or additional cell types) such that vasculogenesis can occur. Pro-vascularization media can comprise appropriate commercially available culture media (e.g., endothelial growth media such as EGM-2MV) optionally supplemented with appropriate cytokines and/or differentiation factors, e.g., VEGF and/or Angiopoietin-1. In some embodiments, vasculogenesis occurs over a period of about 3 to about 7 days by culturing in appropriate media, e.g., vasculogenic media. After vasculature has formed, devices are ready to be assayed by various methods described hereinbelow.

In any of the foregoing embodiments, the BMOC devices further comprise adequately sized media reservoirs (e.g., 3-4 mm) for long-term culture of cells, both within the central channel during monolayer formation and after the addition of gels to the device. The BMOC devices can also further comprise small (e.g., 1 mm) access ports in one or more channels to enable loading and/or handling of small liquid volumes.

In some embodiments of any of the foregoing, the BMOC device can be made of polydimethylsiloxane (PDMS) and can be fabricated according to standard soft lithography techniques. However, any suitable materials can be used to fabricate the BMOC devices, such as polymers (e.g. PDMS, polycarbonate), ceramics (e.g. glass), semiconductors (e.g. silicon) and metal, and combinations thereof. Any suitable fabrication methods can be used to make the devices, such as for example and not limitation, deposition and electro-deposition, etching, bonding, injection molding, embossing and soft lithography.

Generally, a device according to any of the foregoing embodiments can be fabricated using any method used in the art, e.g., soft lithography techniques, to make a device layer. The device layer can be plasma bonded to an upper media reservoir layer, and to a lower coated surface to create the device. In some embodiments, the lower surface is coated with a hydrophobic material, e.g., PDMS. In some embodiments, one or more of the channels comprise hydrophobic surfaces within the channels. In some embodiments, the channels comprising the hydrophobic surfaces are gel and/or media channel(s) and combinations thereof (e.g., one gel channel and one media channel). The hydrophobic surfaces enable the formation of an air-liquid interface between the channels, e.g., between the central channel(s) and the immediately adjacent channel(s). Without wishing to be bound by theory, it is hypothesized that the hydrophobic nature of the channel(s) prevents leaking of fluid from the central channel to the adjacent media channels during extended culture and enables isolated loading of hydrogel into the central channel on top of the cell monolayer.

### Methods of Forming the Distinct BM Microenvironments

As discussed hereinabove, the BMOC devices may comprise previously differentiated cells or undifferentiated cells in the central gel-coated channel(s), which serve as the basis for developing the microenvironments present within BM.

The first step in forming the microenvironments within the BMOC is the formation of the endosteal niche. Here, cells are cultured within the central channel(s) coated or treated with adhesion-promoting materials for period of about 7 to about 21 days to form the endosteal surface. Cells suitable for forming the endosteal niche include but are not limited to MSCs (including human MSCs), pre-osteoblasts, osteoblasts, and/or osteoclasts, and combinations thereof. The central channel(s) can be coated with one or more adhesion-promoting substances including but not limited to polydopamine, fibronectin, fibrin, collagen (e.g., collagen type I), gelatin, polygelatin, other ECM proteins or peptides, and/or ECM-like proteins or peptides, and combinations thereof (e.g., a fibrin-collagen co-gel). In some embodiments, the surfaces of the central channel(s) can be chemically modified, e.g., with a silane such as 3-aminopropyl)triethoxy silane (APTES). The adhesion-promoting substance can also be artificial, e.g., PEG functionalized to promote adhesion. During the culture period, the culture media can comprise commercially available differentiation media (e.g., Stempro Osteo) or alphaMEM supplemented with FBS, dexamethasone, ascorbic acid, beta-glycerophosphate and/or other osteogenic chemicals, cytokines, or additives, as well as other differentiation or growth factors.

Once the endosteal surface has formed, pro-vascularization media and cells can be introduced into the BMOC device to form the central marrow and/or periosteal niche, and/or to promote self-assembly into 3D tissues. To promote formation of these microenvironments or 3D structures, a gel suspension comprising cells and/or tissue is loaded into the central gel-coated channel(s) on top of the endosteal layer. The cells that can be included in the gel suspension include but are not limited to (i) endothelial cells (e.g., HUVECs, MVECs, EPCs, other human endothelial cells, and combinations thereof), (ii) stromal cells (e.g., MSCs (hMSCs) and/or adipocytes), (iii) hematopoietic cells (e.g., HSCs, HPCs, HSPCs, macrophages, neutrophils and combinations thereof), and/or (iv) whole tissue (e.g., processed or unprocessed bone marrow aspirate and/or peripheral blood). The gel can comprise natural substances (e.g., fibrin, fibrinogen, thrombin, collagen, hyaluronic acid, and combinations thereof) and/or artificial polymers (e.g., PEG including functionalized PEG). In some embodiments, endothelial cells, stromal cells, hematopoietic cells, and/or whole tissue, optionally in combination with other cell types such as adipocytes, macrophages, and/or other hematopoietic cells, can be suspended in a gel (a nonlimiting example is a fibrin-based gel) and then loaded on top of the “endosteal” surface in the central gel-coated channel(s). In some embodiments, MSCs (e.g., from bone marrow or other tissue sources), fibroblasts (e.g., from dermal, lung, or other tissue sources), or other cells from a pro-vasculogenic primary cell or cell line can be suspended in a gel (a nonlimiting example is a fibrin-based gel) and loaded into the outer gel-coated channel(s). In some embodiments, the media channel(s) can be filled with appropriate media (e.g., culture media, differentiation media, and/or pro-vascularization media comprising differentiation factors, cytokines, growth factors and/or additional cell types) such that vasculogenesis can occur. Pro-vascularization media can be introduced into the central gel-coated channel(s), the media channel(s), and/or the outer gel-coated channel(s), and can comprise appropriate culture media (e.g., endothelial growth media such as EGM-2MV) optionally supplemented with appropriate cytokines and/or differentiation factors, e.g., VEGF and/or Angiopoietin-1. In some embodiments, the outer gel-coated channel(s) can further comprise cells that promote vasculogenesis, such as for example and not limitation, MSCs or human MSCs (e.g., derived from bone marrow or other tissue sources), fibroblasts (e.g., derived from dermal, lung, or other tissue sources), and/or a pro-vasculogenic primary cell or cell line. The BMOC devices are cultured in vasculogenic media for about 3 to about 7 days prior to assay or use as described hereinbelow.

### Clinical and Diagnostic Methods of the Disclosure

The microfluidic BMOC devices described herein can be used with a variety of clinical and diagnostic methods. For example and not limitation, the effects of different therapies (e.g., chemotherapy, radiation therapy), therapeutics (e.g., biologic drugs including proteins, DNA, RNA, lipids, and/or cells; small molecule drugs), and cell-based therapies (e.g., human stem cell (HSC) therapies, CAR T cell therapies) on different cells and microenvironments within BM (e.g., human BM) can be determined. In some embodiments, the cells used in the BMOC devices are from well-known cell lines. In some embodiments, the cells are models for certain diseases, e.g., certain cancer types or hematopoietic syndromes. In some embodiments, the cells are derived from primary cells obtained from certain patients, e.g., MSCs and HSCs (e.g., from bone marrow aspirates or other tissue samples), in order to provide patient-specific results.

In some embodiments, mobilization of the bone marrow, including stem cells within the bone marrow, can be studied using the BMOC devices of the disclosure. In some embodiments, the mobilization can be studied to determine the effects of certain therapies or treatments, including but not limited to cancer treatment (e.g., chemotherapy and/or radiation therapy). In some embodiments, the BM stem cell mobilization is measured by collecting the stem cells via leukophoresis.

In some embodiments, BM stem cells are used to generate BMOC devices to generate a complex bone marrow tissue model for preclinical screening of therapeutics (small molecules, biologics). The BM stem cells can be derived from well-known cell lines to create a generic BMOC device, or from a sample taken from a patient (such as for example and not limitation bone marrow aspirate, tissue sample or MSCs) to generate a patient-specific BMOC device.

In some embodiments, the effects of accidental or therapeutic radiation (e.g., as part of cancer treatment) on BM microenvironments can be studied using BMOC devices of the disclosure. The devices can also be used to study potential countermeasures to treat or mitigate effects from such exposure, including for example and not limitation, the use of therapeutic small molecules, biologics (nonlimiting examples include proteins, DNA, RNA, lipids, or cells), and/or MSCs.

In some embodiments, the BMOC device can be used to recreate a cancer or tumor microenvironment in BM. For example and not limitation, hematopoietic malignancies (nonlimiting examples include leukemia, lymphoma, myeloma) primary samples (e.g., patient-derived primary samples such as bone marrow aspirate, tissue samples, or MSCs) or cell lines can be introduced into the central gel-coated channel(s) of the BMOC device to provide a model of the malignancy. In other embodiments, the BMOC device can be used to recreate other malignancies (nonlimiting examples include breast cancer, prostate cancer, lung cancer, liver cancer, melanoma, head and neck cancer, gastrointestinal cancer, ovarian cancer, cervical cancer) from primary samples (e.g., patient-derived primary samples such as bone marrow aspirate, tissue samples, or MSCs) or cell lines can be introduced into the central gel-coated channel(s) of the BMOC device to provide a model of the malignancy.

In some embodiments, the BMOC device can be used to form a tissue specific cancer model for preclinical screening of therapeutics (e.g., chemotherapy, radiotherapy, immunotherapy, cell-based therapies) in a generic or patient-specific format, depending on the source of the cells used to form the BMOC (e.g., from a cell line or from a sample taken from the patient such as for example and not limitation bone marrow aspirate, tissue sample or MSCs).

In any of the foregoing embodiments, the BMOC devices can be assayed by one or more methods involving measurement of cytokines, microscopy, assaying cell death or killing, and/or recovery of cells and/or gels from the BMOC device.

For example and not limitation, cytokines produced by cells in the BMOC device can be collected and measured (e.g., identified and/or quantified) by any method commonly known in the art, including measurement by ELISA and multiplex cytokine detection (Luminex).

The BMOC devices can be assayed by a number of different microscopic methods, including immunohistochemistry (which enables staining for specific cell markers, ECM, cytokines, and other features of the BMOC), cell tracking (which involves pre-labeling cells for live imaging and measurement of cell movement and behavior relative to the different niches in the BMOC), vascular network analysis (which enables measurement of the effects of treatments on vascular networks (e.g., by programs such as Angiotool and ImageJ), and/or determining cell engraftment into the different BM niches.

In some embodiments, cell death and/or killing within the BMOC device can be determined by, for example and not limitation, the measurement of specific cell death (e.g., by TUNEL and similar methods) or bulk cytotoxicity (e.g., assaying for LDH).

In some embodiments, the gel from the gel-coated channel(s) and/or cells can be removed from the BMOC device for further assaying. In some embodiments, single cells can be assayed, e.g., by RNA-seq, flow cytometry, and other known methods. In some embodiments, protein expression or nucleic acid expression can be measured by any method known in the art, e.g., Western blotting, ELISA, quantitative PCR.

### EXAMPLES

The present disclosure is also described and demonstrated by way of the following examples. However, the use of these and other examples anywhere in the specification is illustrative only and in no way limits the scope and meaning of the disclosure or of any exemplified term. Likewise, the disclosure is not limited to any particular preferred embodiments described here. Indeed, many modifications and variations of the disclosure may be apparent to those skilled in the art upon reading this specification, and such variations can be made without departing from the disclosure in spirit or in scope. The disclosure is therefore to be limited only by the terms of the appended claims along with the full scope of equivalents to which those claims are entitled.

### Example 1. Development of a Three Channel BMOC Device

A three-channel polydimethylsiloxane (PDMS) microfluidic BMOC device was designed and fabricated using standard soft lithography techniques (FIG. 1). The central channels of the devices were coated with polydopamine and type I collagen to promote cellular adhesion to the PDMS surface. BM human mesenchymal stem cells (BM-hMSCs) (RoosterBio Inc) were separately expanded and differentiated toward osteoblasts (OBs) to serve as stromal cells in the central marrow and endosteal niche, respectively. After 14 days of differentiation, hOBs were seeded at a high density in the central channel of the human BMOC. OBs were cultured for an additional 10 days within the device to allow for mineralization and the establishment of the endosteal niche. To observe mineralization of the endosteal niche, devices were fixed and stained with Alizarin Red. hMSCs (1×10⁶ cells/mL) and HUVECs (5×10⁶ cells/mL) were resuspended in a thrombin (2 U/mL) and fibrinogen (5 mg/mL) solution and flowed in to the central channel of the device, where a fibrin gel was formed. The human BMOC device was cultured for 5 days in media supplemented with VEGF and Angiopoietin-1 (Peprotech). Expression of cytokines (SCF, SDF1, Jagged-1) and extracellular matrix (ECM) (fibronectin, laminin, osteopontin) components of the BM microenvironment were observed in the endosteal niche, perivascular niche, and complete human BMOCs using immunofluorescence. Fluorescently labeled CD34+BM cells (Lonza) were loaded into hBM-on-a-chip and flowed through the vasculature; the human BMOC was imaged using a fluorescence confocal microscope and number of engrafted CD34+ cells were counted at various time points.

Results: OBs adhered and proliferated on polydopamine and collagen type I coated PDMS surfaces within human BMOC over 10 days. Matrix mineralization was observed by Alizarin Red staining and expression of SDF1, Jagged-1, SCF, and fibronectin were observed by fluorescence microscopy. Vasculogenesis was observed during 5 days of HUVEC/MSC culture within the human BMOC. The vascular network was perfusable by 70 kDa FITC-dextran as well as 8 μm polystyrene beads. Using fluorescence microscopy, it was found that endothelial and perivascular cells expressed SCF, SDF1, and fibronectin.

Conclusions: The described microfluidic device, or human BMOC, effectively recapitulates the basic organization and characteristics of the endosteal and perivascular niches found in BM. It is suggested that this device can be used to study engraftment and mobilization of hHSCs to and from the BM niche.

### Example 2. Development of a Five Channel BMOC Device

### Methods

### Photomask Design

Photomasks (CAD/Art Services) were designed using AutoCAD software (Autodesk). Photomasks were specified as right-read, darkfield, emulsion down.

### Soft Lithography

An SU-8 master mold was fabricated using previously described soft lithography techniques. Briefly, SU-8 2150 (MicroChem) was spun to a thickness of 120 μm on a silicon wafer (University Wafers) using a G3P8 Spin Coater (SCS). SU-8 was exposed with UV light through the photomask using an MJB4 mask aligner (Suss Microtec). Uncrosslinked SU-8 was removed with SU-8 developer (MicroChem) and silicon wafers were treated by vapor phase deposition of trichloro(1H,1H,2H,2H-perfluorooctyl silane (Sigma-Aldrich) to increase surface hydrophobicity.

### PDMS Device Fabrication

### Version 1.0 Device Fabrication

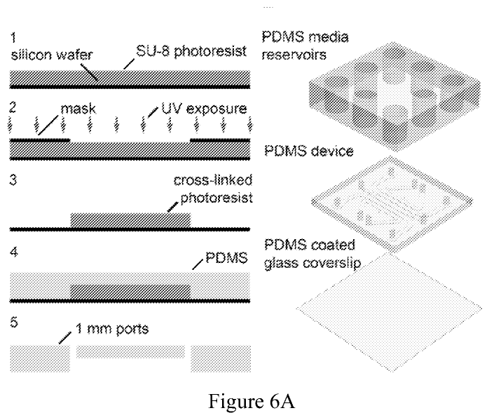

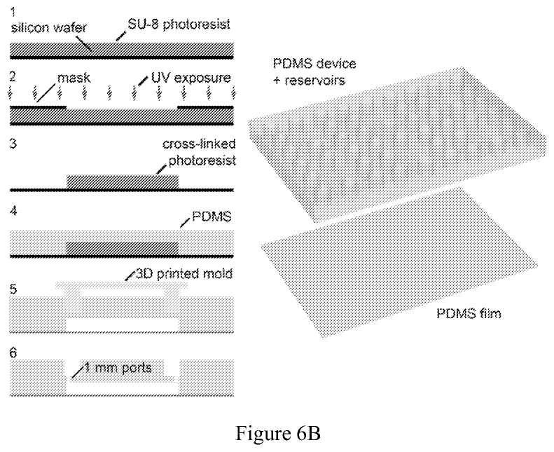

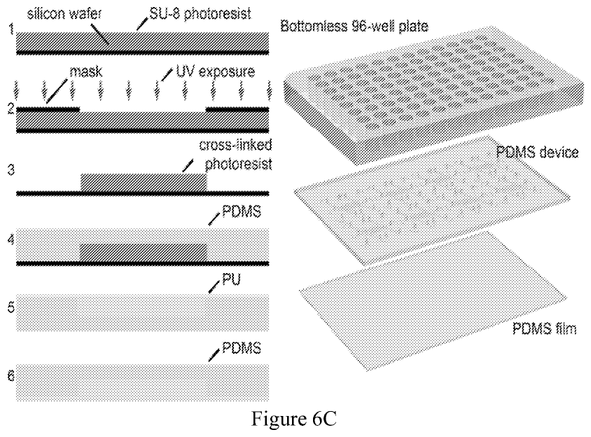

PDMS (Dow Corning) was mixed 10:1 (elastomer base: curing agent) and cast on SU-8 master mold. PDMS was cured at 65° C. The PDMS layer containing the features was then removed from the master mold and loading ports were made using a 1 mm biopsy punch (Integra Miltex). To form the media reservoir layer, PDMS was mixed 10:1 and cast in a 100 mm petri dish to a thickness of 5-6 mm. PDMS was cured at 65° C. The thick disc of PDMS was then cut into single-device sized squares and media reservoirs were made using a 4 mm biopsy punch. The top surface of the device layer was then bonded to the media reservoir layer using a plasma cleaner (Harrick Plasma). To form the PDMS coated coverslips, PDMS was mixed 10:1 and 50 μL were applied to a clean glass coverslip, the coverslip was then sheared against a glass slide to evenly coat the surface and the PDMS was cured at 65° C. The PDMS coated coverslip was then bonded to the bottom surface of the device layer using a plasma cleaner (FIG. 6A). Prior to use, devices were washed with 70% EtOH.

### Version 1.1 Device Fabrication

PDMS was mixed 10:1 and cast on SU-8 master mold. PDMS was cured at 65° C. The PDMS layer containing the features was then removed from the master mold, a 3D printed reservoir mold was aligned on top and additional PMDS (10:1) was poured on top of the device to form media reservoirs. After curing at 65° C., loading ports were made using 1 mm biopsy punch. A thin film of PDMS was made by mixing PDMS 5:1, casting a thin layer (˜300 μm) in a 150 mm petri dish and curing at 65° C. Devices were bonded to the thin film of PDMS using a plasma cleaner, and individual devices were cut for use in cell culture (FIG. 6B). Prior to use, devices were washed with 70% EtOH.

### Version 2.0 Device Fabrication

hBM-on-a-chip was integrated into a standard well-plate format using previously published methods. Because a standard 100 mm silicon wafer does not have sufficient area to pattern the entire 4×2 hBM-on-a-chip array (FIG. 6C), a polyurethane master mold was first fabricated from PDMS cast on a SU-8 master. PDMS was mixed 10:1 (elastomer base: curing agent) and cast on the SU-8 master mold 0 to create two copies of the 2×2 array. The two pieces were aligned, feature-side down, and made into a single block by casting in additional PDMS. A polyurethane master mold was then cast on the single PDMS piece containing the 4×2 array using a 2-part polyurethane liquid plastic (Smooth Cast 310, Smooth-On Inc). To prevent adhesion of PDMS subsequently cast, the polyurethane master mold was treated by vapor phase deposition of trichloro(1H,1H,2H,2H-perfluorooctyl) silane (Sigma-Aldrich).

PDMS was mixed 10:1, cast on the polyurethane master mold, and cured at 65° C. The PDMS layer containing the features was then removed from the master mold and loading ports were made using a 1 mm biopsy punch. A thin film of PDMS was made by mixing PDMS 5:1, casting a thin layer (˜600 μm) in a 150 mm petri dish and curing at 65° C. The feature layer of PDMS was then bonded to the PDMS film using a plasma cleaner. The bonded PDMS devices were then attached to a bottomless 96-well plate (Greiner Bio-One) using a chemical gluing method. Briefly, the 96-well plate was immersed in 2% (v/v) 3-mercaptopropyl trimethoxysilane (Sigma-Aldrich) in methanol for 1 minute, rinsed with deionized H₂O, and dried. The bonded PDMS devices were then plasma bonded to the 96-well plate using a plasma cleaner. To provide support to the bottom PDMS surface, glass coverslips (Fisher Scientific) were adhered to the PDMS film by plasma bonding. Prior to use, devices were washed with 70% EtOH and DI H₂O.

### Burst Pressure Calculations

Burst pressure was calculated using an approached described by Wang et al. 2016, which is briefly described below. The pressure difference at the air-liquid interface for advancing liquid within the gel channel can be represented by the Young-Laplace equation (Equation 1):

$${P}_{liquid advance} - {P}_{air} = - 2 γ ( \frac{cos {θ}_{A}}{{w}_{c h a n n e l}} + \frac{cos {θ}_{A}}{h} ) \qquad ( 1 )$$

where P_{liquid advance} is the liquid pressure inside the gel channel, γ is surface tension (γ=0.072 N m⁻¹), θ_{A} is the critical contact angle where liquid will burst or advance (θ_{A}=140°), w is the width of the channel, and h is the height of the channel. The advancing pressure for liquid within gel channel (w_{channel}=1000 μm, h=150 μm) was found to be 845 Pa or 86 mmH₂O.

The burst pressure at the air-liquid interface at the pores is represented in Equation 2:

$${P}_{liquid burst} - {P}_{a i r} = - 2 γ ( \frac{cos {θ}_{A}^{*}}{{w}_{p o r e}} + \frac{cos {θ}_{A}}{h} ) \qquad ( 2 )$$

where θ_{A}* is the contact angle of the liquid with the inner facing side wall of the channel dividers (however, θ_{A}* is limited to 180°, the maximum contact angle for a liquid meniscus). The burst pressure at the communication pores (wₚₒᵣₑ=50 μm, h=150 μm) is 3615 Pa or 369 mmH₂O. The difference between P_{liquid advance} and P_{liquid burst} is 2770 Pa or 282 mmH₂O.

### Cell Culture

### Mesenchymal Stem Cells (MSCs)

Bone marrow derived human MSCs (RoosterBio) were initially expanded in hMSC High Performance Media (RoosterBio) for a single passage. For subsequent passages, hMSCs were expanded in aMEM (Sigma-Aldrich) supplemented with 10% FBS (Hyclone) and 1% penicillin-streptomycin (Hyclone). hMSCs were used for culture in devices up to passage 6.

### Human Umbilical Vein Endothelial Cells (HUVECs)

HUVECs (Lonza) were expanded in EBM-2MV (Lonza) on tissue culture flasks coated with 0.1% gelatin (Sigma-Aldrich) and used up to passage 8.

### Surface Coating of PDMS

To promote cell adhesion, the central gel channel was coated with 0.01% dopamine HCl (Sigma-Aldrich) in TE Buffer [pH 8.5] for 1 hour at room temperature, washed with PBS, coated with 100 μg/mL rat tail collagen I (Corning) in PBS for 1 hour at room temperature, washed with PBS, and then dried overnight at 65° C. Devices were sterilized by UV exposure for at least 30 minutes prior to culture of cells.

### Vasculogenesis

Vasculogenesis in central gel-coated channel was accomplished using previously reported approaches. Briefly, HUVECs (12×10⁶ cells/mL) and MSCs (6×10⁵ cells/mL) were suspended in EBM-2MV supplemented with human thrombin (4 U/mL) (Sigma-Aldrich). Solutions of human fibrinogen (Sigma-Aldrich) and/or rat tail collagen I (Corning) were prepared at twice the final concentration in PBS as indicated in the figure. The HUVEC/MSC cell suspension was added to fibrinogen solution (1:1) and mixed thoroughly. (Final cell suspension: HUVECs (6×10⁶ cells/mL), hMSCs (3×10⁵ cells/mL), thrombin (2 U/mL), fibrinogen (variable), collagen I (variable)). Immediately, the hMSC/HUVEC cell suspension was withdrawn from the opposite central gel port, drawing the cell suspension through the central gel channel. hMSCs were similarly loaded into the outer channels of the device. (Final cell suspension: hMSCs (3×10⁶ cells/mL), thrombin (2 U/mL), fibrinogen (3 mg/mL). Devices were then incubated for 15 minutes at 37° C., 5% CO₂ to allow the fibrin gel to form. EBM-2MV was added to a reservoir on each side of gel channel and pulled through media channel, into connecting reservoir by using a micropipette to create negative pressure in the connecting 1-mm port. Cells were cultured with daily media exchange for 5 days to allow for vasculogenesis. Media was supplemented as indicated. VEGF (50 ng/ml) was supplemented on days 2-5, angiopoietin-1 (100 ng/mL) was supplemented on days 3-5.

### Perfusion

Five days after initial cell seeding, perfusion of the vasculature was tested by flowing fluorescein isothiocyanate (FITC)-dextran (MW 70 kDa, Sigma-Aldrich). Media was aspirated from the device media reservoirs and 50 μL 10 μg/mL FITC-dextran in EBM-2MV was added to a single reservoir. Subsequent perfusion of the vascular network was visualized using a Lionheart FX (Biotek Instruments).

### Cell Culture

### Human Bone Marrow CD34+ Hematopoietic Stem and Progenitor Cells (HSPCs)

Human BM CD34+ cells (Lonza) were expanded for 5 days in Stemline II (Sigma-Aldrich) supplemented with 100 ng/mL SCF, TPO, and G-CSF (Peprotech). All cells were cultured at 37° C. and 5% CO₂.

Osteogenesis in hBM-On-a-Chip

For the formation of the endosteal niche, hMSCs were seeded within the central gel channels of devices at a density of 5×10⁵ cells/mL. Cells were cultured within the devices for 21 days in αMEM osteogenic media (10% FBS, 1% penicillin-streptomycin, 10 mM (3-glycerophosphate (Sigma-Aldrich), 50 μM ascorbic acid (Sigma-Aldrich), and 100 nM dexamethasone (Sigma-Aldrich)) with daily media exchange.

### Alizarin Red and Von Kossa Staining and Quantification

Osteogenic devices were washed with PBS, fixed with 4% formaldehyde in PBS for 15 minutes, and then washed with PBS. For Alizarin red staining, devices were washed twice with DI H₂O and then stained for 5 minutes with 2% alizarin red (Sigma-Aldrich) in DI H₂O [pH 4.1-4.3]. Alizarin red stain was removed by several washes with DI H₂O, until liquid was clear. For von Kossa staining, devices were washed twice with DI H₂O and then stained with 1% silver nitrate (Acros Organics) in DI H₂O 2O under a UV lamp for 15 minutes. Devices were washed twice with DI H₂O and then incubated in 5% sodium thiosulfate (Acros Organics) in DI H₂O 2O for 5 minutes. Devices were then washed with DI H₂O 2O until liquid was clear.

Stained devices were imaged using a Lionheart FX (BioTek Instruments). Color brightfield images were analyzed using open-source software ImageJ (https://imagej.nih.gov/ij/index.html). The red and green channels were used for von Kossa and Alizarin, respectively, to measure mean intensity and percent area.

Vasculogenesis in hBM-on-a-chip

Vasculogenesis in central gel channel was accomplished using previously reported approaches. Briefly, HUVECs (12×10⁶ cells/mL) and MSCs (6×10⁵ cells/mL) were suspended in EBM-2MV supplemented with thrombin (4 U/mL) (Sigma-Aldrich). A solution of fibrinogen (8 mg/mL) (Sigma-Aldrich) and collagen I (2 mg/mL) (Corning) in PBS was loaded into a central gel channel reservoir. The HUVEC/MSC cell suspension was added to fibrinogen solution (1:1) and mixed thoroughly. (Final cell suspension: HUVECs (6×10⁶ cells/mL), hMSCs (3×10⁵ cells/mL), thrombin (2 U/mL), fibrinogen (5 mg/mL)), collagen I (1 mg/mL). Immediately, the hMSC/HUVEC cell suspension was withdrawn from the opposite central gel port, drawing the cell suspension through the central gel channel. Devices were then incubated for 15 minutes at 37° C., 5% CO₂ to allow the fibrin gel to form. EBM-2MV was added to a reservoir on each side of gel channel and pulled through media channel, into connecting reservoir by using a micropipette to create negative pressure in the connecting 1-mm port. Cells were cultured with daily media exchange for 5 days to allow for vasculogenesis. Media was supplemented with VEGF (50 ng/mL) on day 2 and with VEGF (50 ng/mL) and angiopoietin-1 (ANG-1) (100 ng/mL) (Peprotech) on days 3, 4, and 5.

### Immunofluorescence Staining and Microscopy

Staining procedure for devices was adapted from previously reported approaches. Devices were washed with PBS, fixed with 4% formaldehyde (ThermoFisher), and permeabilized with 0.1% Triton X-100. Prior to staining, cells were blocked with 5% BSA, 3% goat serum in PBS. Primary antibodies were diluted (1:100) in blocking buffer and devices were stained overnight at 4° C. Devices were then washed with 0.1% BSA in PBS and stained with secondary antibodies (1:200) diluted in wash buffer for 3 hours at RT. Devices were washed with wash buffer and then stained with DAPI (1:1000) and Phalloidin AF647 (1:40).

### Vascular Network Analysis

Devices stained with Alexa Fluor 647 anti-human CD31 (BioLegend) and Alexa Fluor 594 Phalloidin (ThermoFisher) were imaged using a Lionheart FX (BioTek Instruments). Images were processed using open-source software ImageJ and contrast corrected images were analyzed using AngioTool to measure percent area and total network length.

### Multiplex Cytokine Detection

Media was collected from devices at designated times by collecting media from one side of device, waiting for 5 minutes to allow for gravity driven flow through the device and then collection of all media from reservoirs. Device media was immediately stored on ice and then flash frozen in liquid N2 for storage prior to analysis. Samples were thawed on ice prior to detection and analysis using LEGENDplex human hematopoietic stem cell panel (BioLegend), according to manufacturer's protocol.

### Expansion of CD34+BM HSPCs

BM CD34+ HSPCs were labelled with PKH67 green fluorescent cell stain (Sigma Aldrich) and loaded at a 20:1 (HUVEC: HSPC) ratio (final concentration 6×10⁵ HSPC/mL) with the endothelial cells and hMSCs. This concentration results in 600 HSPCs within the central channel. Devices were imaged on days 0, 1, 3, and 5 after loading using a Lionheart FX (BioTek Instruments). Images were analysed using Gen5 (BioTek Instruments) to count the number of HSPCs and progenitors at each time point.

### Statistical Analysis

Sample sizes and statistical methods are indicated in the figure captions and individual data points are shown. GraphPad Prism was used for statistical analysis. Normality of samples were tested using Shapiro-Wilk normality test. If all samples in an experiment passed normality test, one-way ANOVA with Tukey's multiple comparison test was used. If a sample did not pass normality test, Kruskal-Wallis with Dunn's multiple comparison test was used.

### Results

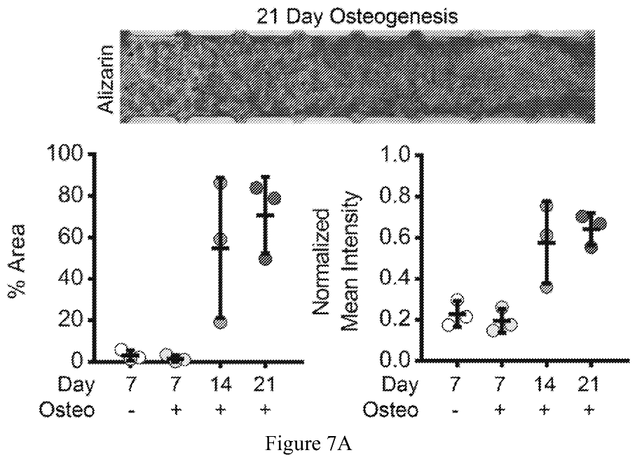

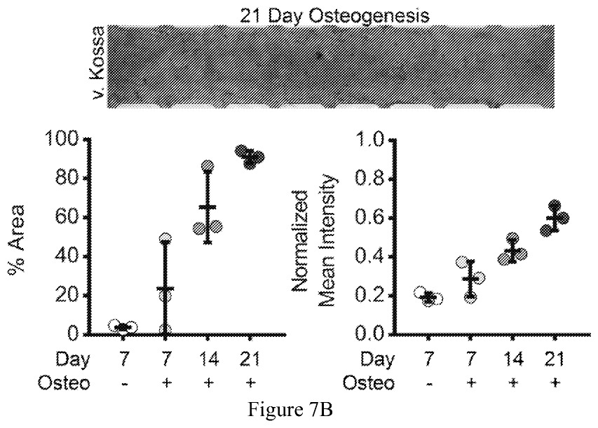

Human MSCs were seeded at a high density within the central channel of the device and differentiated with osteogenic media over a period of 21 days. Mineralization of matrix was observed by Alizarin red (FIG. 7A) and von Kossa (FIG. 7B) staining. Mineralization increased over the 21-day differentiation. At 21 days, 71±18% of the area of the device stained positive for Alizarin red and the normalized mean intensity of the stained device was 0.64±0.07. Similarly, at 21 days 91±3% of the area of the device stained positive for von Kossa and the normalized mean intensity was 0.60±0.06. In addition to mineralized matrix, the expression of specific cytokines and ECM components is of interest when creating a surrogate endosteal niche. After 21 days differentiation, the differentiated hMSCs expressed cytokines (SCF, CXCL12, JAG1) and ECM (FN, OPN) characteristics of the endosteal niche.

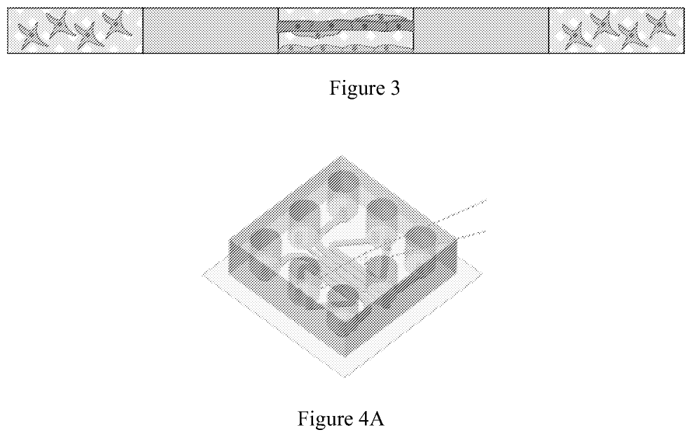

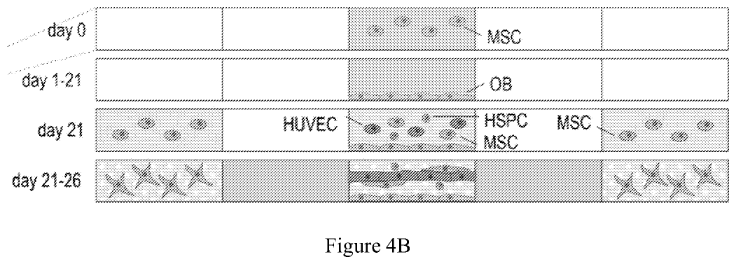

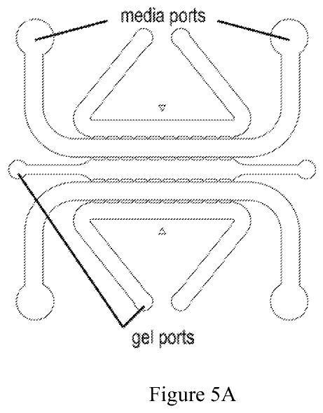

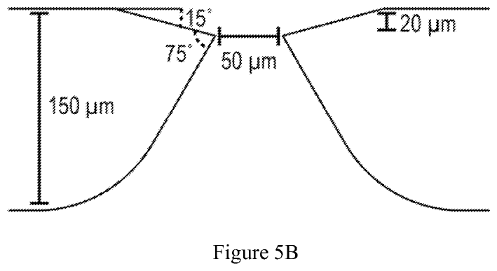

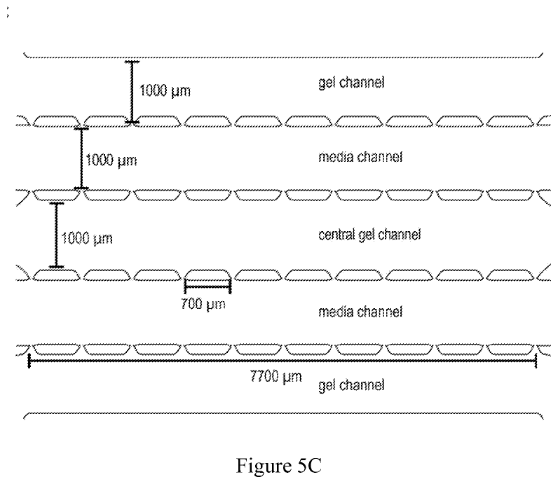

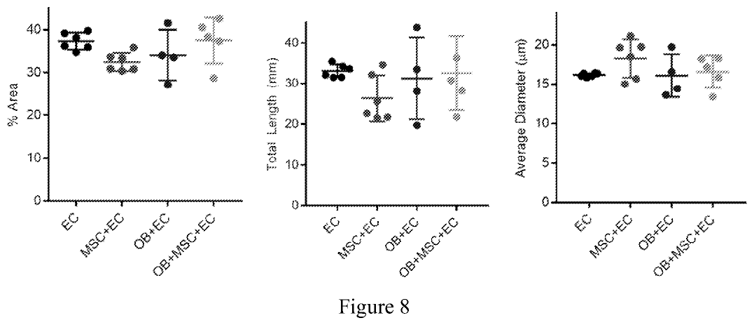

Once the endosteal niche was formed, HUVECs and hMSCs were suspended in a fibrin (5 mg/mL) hydrogel and loaded on top of the endosteal surface. Using a 5-channel device (FIGS. 3-5), hMSCs were simultaneously loaded into the outermost channels in order to produce pro-vasculogenic cytokines and induce vascular formation. To determine the effect of stromal cells on vasculogenesis, devices were made with and without hMSCs and hOBs and measured the resulting vascular formation (FIG. 8). F-actin staining of all cells (endothelial cells were differentiated by CD31 expression) showed perivascular stromal cells in MSC containing conditions and an endosteal layer in OB containing groups. Qualitatively, the resulting vascular networks appeared different. EC-only vasculature appeared larger and less homogeneous, while MSCs and OBs appeared to produce more consistent vasculature and decrease the vessel diameter. However, analysis of the vascular networks did not find any significant differences between groups for either the percent area or total lengths of the networks (FIG. 8).

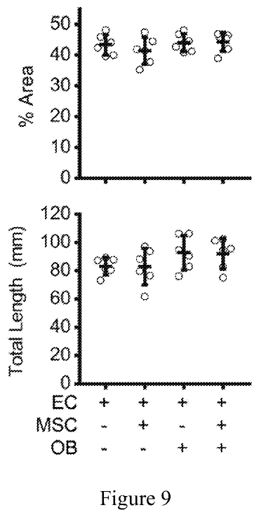

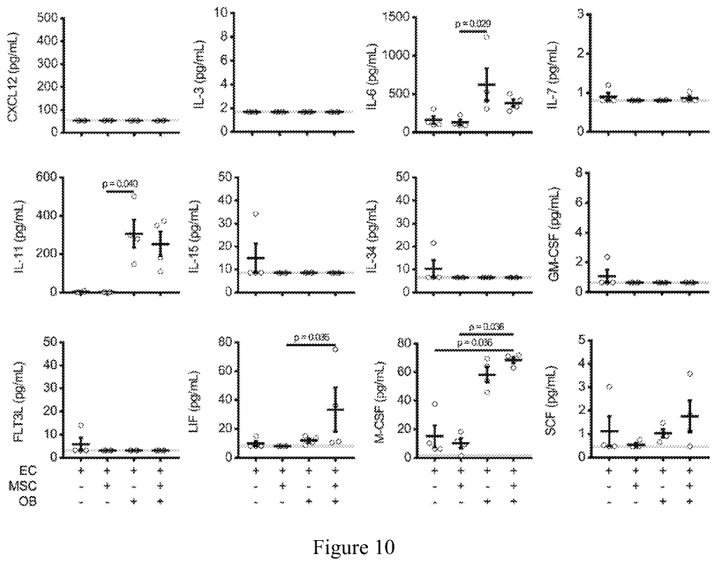

Using the fibrin-collagen I co-gel with supplemental cytokines (VEGF and Ang-1), human BMOC devices were created with and without hMSCs and the differentiated endosteal layer (OBs) to measure the effect of stromal cells on vasculogenesis and cytokine secretion (FIGS. 9 and 10). No significant difference was observed in the vasculature area containing hMSCs (41.4±4.4%), OBs (44.1±2.9%), or both cell types (44.3±3.1%) when compared between groups or to devices containing ECs only (43.39±3.3%) (FIG. 9). Similarly, no significant difference in the total length of the vasculature networks was observed between devices containing hMSCs (83.0±13.0 mm), OBs (92.9±12.2 mm), both hMSCs and OBs (92.1±10.8 mm), or neither (83.3±6.2 mm) (FIG. 10). Although there was no significant difference, it is worth noting that the devices containing OBs exhibited vasculature that covered marginally less area and had a slightly increased total length of the network, which indicated a smaller average diameter of the newly formed vasculature compared to devices without OBs.

Differences in cytokine expression were observed as a function of stromal cell inclusion using multiplex cytokine detection to analyze the supernatant collected from the devices on day 5 of vasculogenesis (FIG. 10). OB containing devices, in general, secreted higher amounts of cytokines. IL-6, a cytokine involved in B cell differentiation, was highly expressed in EC+OB (624±212 pg/mL) and EC+MSC+OB (386±49 pg/mL) samples, less was measured to be present in EC (162±49 pg/mL) and EC+MSC (132±34 pg/mL). IL-11, which is responsible for signaling during megakaryocyte maturation, was measured in trace concentrations in EC (4.1±1.8 pg/mL) devices and not at all in EC+MSC samples, while substantial concentrations were observed in EC+OB (308±73 pg/mL) and EC+MSC+OB (254±64 pg/mL) devices. Similarly, M-CSF, which induces macrophage differentiation of HSCs, was elevated in EC+OB (58.3±5.3 pg/mL) and EC+MSC+OB (68.6±2.1 pg/mL) devices, while little was detected in EC (15.2±7.6 pg/mL) and EC+MSC (10.3±3.5 pg/mL) devices.

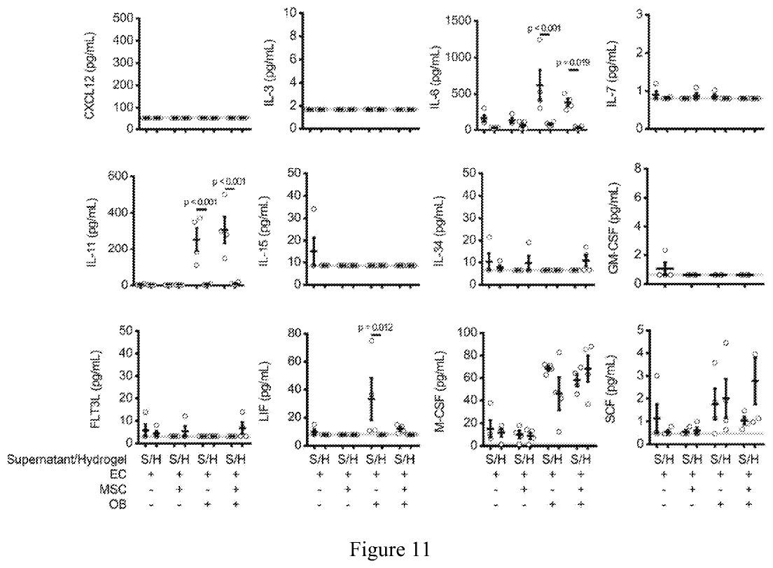

Relatively small concentrations of IL-7 (promoter of lymphopoiesis), IL-34 (monocytes and macrophages), GM-CSF (granulocytes and macrophages), FLT-3L (dendritic cells), and SCF (HSC maintenance) were measured and there was no significant difference across groups. For both CXCL12 (hematopoietic chemoattractant) and IL-3 (myeloid progenitors), no measurable analytes were detected. A limitation of microfluidic cell culture is the small volume of media that is accessible to the cells and the small number of cells in each culture, which can result in low (and in this case below detection) concentrations of analytes. To circumvent this issue and obtain more concentrated samples, the inventors were also able to recover the central hydrogel and assay the cell containing portion of the device directly by cutting through the bottom PDMS film and retrieving the central hydrogel. However, this approach resulted in no increase in the recovery of soluble cytokines (FIG. 11).

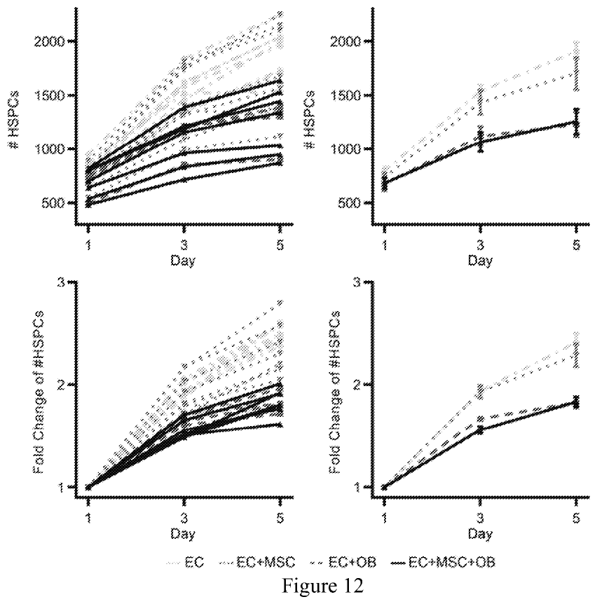

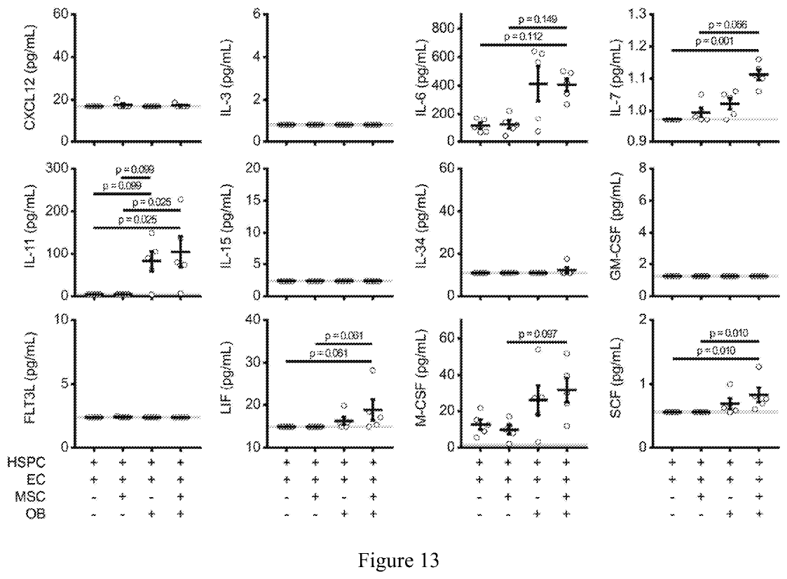

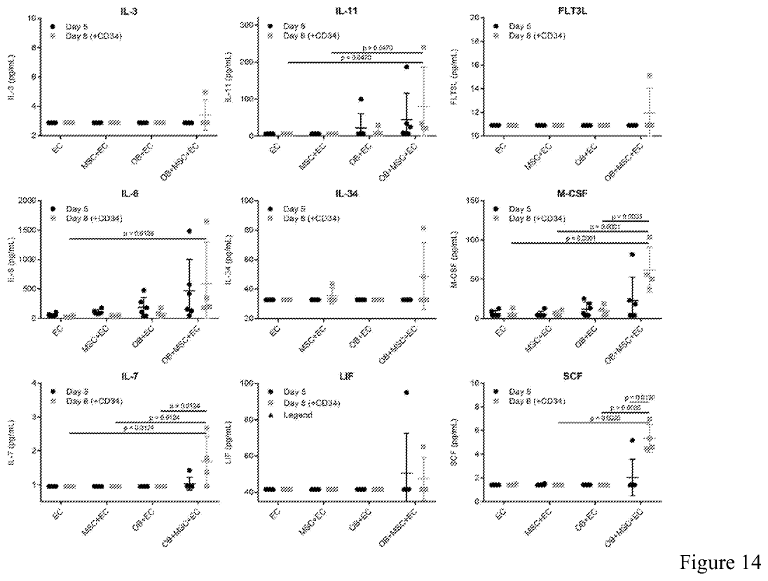

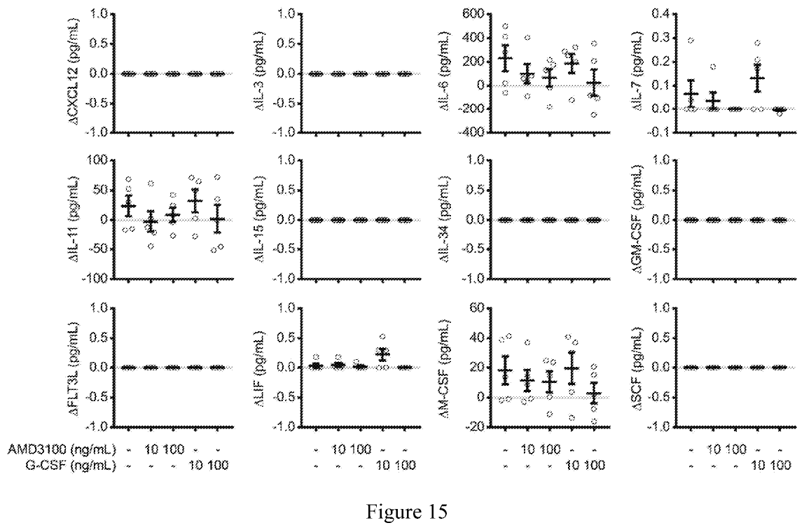

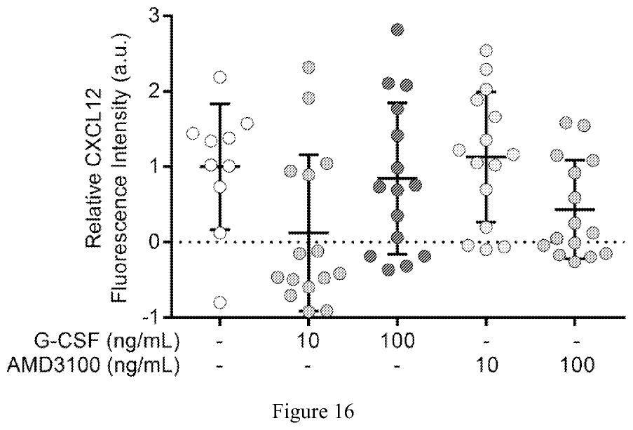

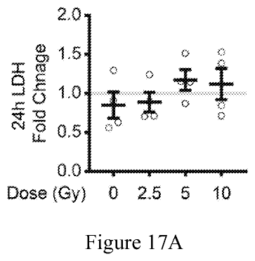

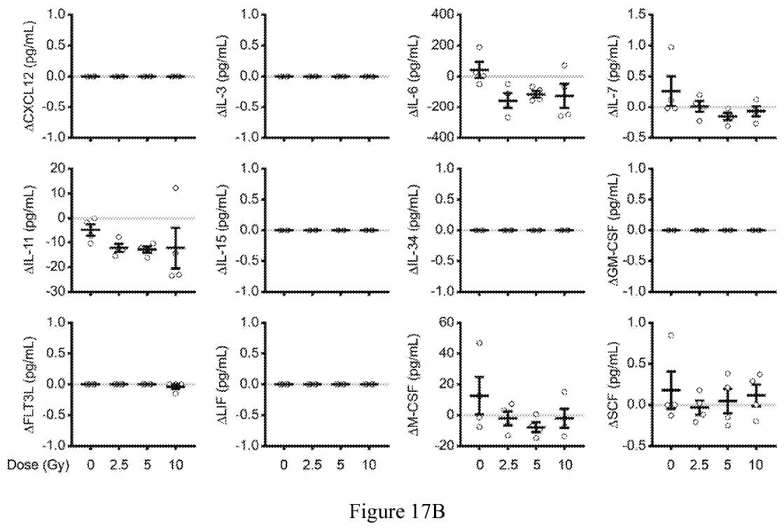

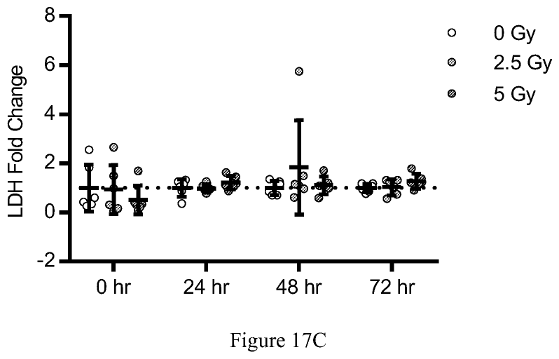

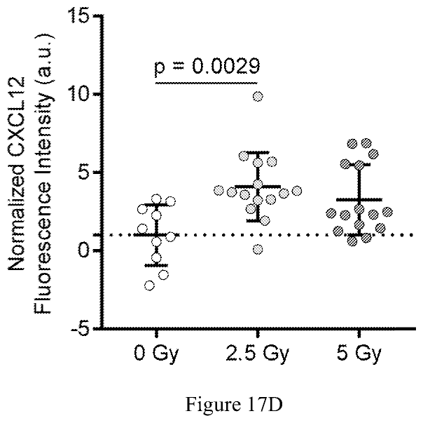

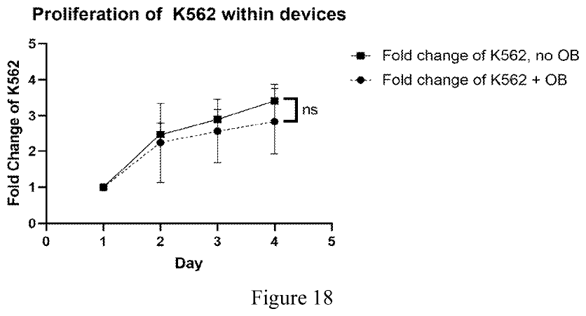

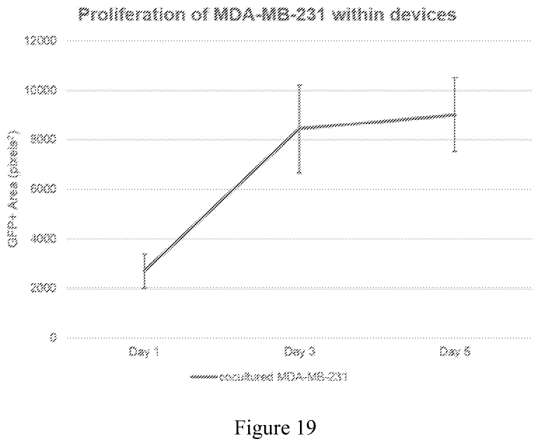

After 5 days of vasculogenesis, human BMOC devices containing the endosteal layer and subsequently seeded hMSCs and HUVECs were fixed and stained to characterize the presence and localization of cytokines and ECM relevant to the BM niche (FIG. 12). CXCL12 and SCF were both found to be expressed by perivascular and endothelial cells. Fibronectin was observed to be present in the “central marrow” space outside of the newly formed vasculature. This arrangement of cytokine expression is consistent with the distribution that has been seen in vivo.

The inventors next sought to investigate the inclusion of BM HSPCs in the human BMOC devices and how hMSCs and OBs affected their fate in the BMOC model. BM CD34+ cells were briefly expanded in vitro out of cryo-storage and then loaded into the central channel with HUVECs and hMSCs. The inventors measured the expansion of the HSPCs via microscopy over the 5 days of culture during vasculogenesis (FIG. 12). On day 1, the number of HSPCs in human BMOC devices was not significantly different in MSC and/or OB containing devices (FIG. 12 top panel). By day 3 and continuing on to day 5, devices without OBs had significantly more HSPCs than devices with the endosteal layer. HSPCs culture with ECs and MSCs expanded 2.41- and 2.28-fold, respectively, over 5 days, whereas both groups with HSPCs cultured in the presence of the pre-formed endosteal layer only expanded 1.83-fold (FIG. 12 bottom panel).

Conclusions: The 5-channel BMOC recreated basic features of the endosteal and perivascular niches of BM. Vasculogenesis occurred in the presence and absence of differentiated osteoblasts. Increased cytokine expression in the presence of osteoblasts suggested that increased complexity is needed to recapitulate the BM microenvironment and correspondingly HSPCs expand at different rates in the presence and absence of the endosteal layer.

### List of Embodiments

Embodiment 1. A multichannel multifluidic bone marrow-on-a-chip (BMOC) device comprising:

at least three channels;

at least one media port; and

at least one gel port,

wherein the at least three channels comprise at least one central channel and at least two external channels,

wherein one external channel is located on one side of the at least one central channel and proximal to the at least one central channel,

wherein another external channel is located on the opposite side of the at least one central channel and proximal to the at least one central channel,

wherein the at least one central channel comprises:
a coating or surface treatment comprising one or more of polydopamine, fibrin, fibrinogen, fibronectin, collagen, hyaluronic acid, extracellular matrix (ECM) proteins or peptides, ECM-like proteins or peptides, PEG and functionalized PEG; and either (i) differentiated cells selected from the group consisting of osteoblasts and osteoclasts; or (ii) undifferentiated cells selected from the group consisting of MSCs, HSCs, HUVECs, pre-OBs, and other MSC-derived bone-lineage cells,

wherein the at least one central channel contains one or more of (i) pro-vascularization media, (ii) a pro-vascularization gel suspension comprising one or more of fibrin, fibrinogen, fibronectin, collagen, gelatin, hyaluronic acid, polyethylene glycol, functionalized polyethylene glycol, and combinations thereof, iii) whole tissue comprising bone marrow aspirate and peripheral blood, and (iv) cells selected from the group consisting of endothelial cells, hematopoietic cells, and stromal cells, and

wherein, prior to addition of the one or more of (i) pro-vascularization media, (ii) a pro-vascularization gel suspension comprising one or more of fibrin, fibrinogen, fibronectin, collagen, gelatin, hyaluronic acid, polyethylene glycol, functionalized polyethylene glycol, and combinations thereof, iii) whole tissue comprising bone marrow aspirate and peripheral blood, and (iv) cells selected from the group consisting of endothelial cells, hematopoietic cells, and stromal cells, the BMOC device is cultured for a period of about 7 to about 21 days to promote formation of the endosteal surface microenvironment of bone marrow,

wherein, after addition of the one or more of (i) pro-vascularization media, (ii) a pro-vascularization gel suspension comprising one or more of fibrin, fibrinogen, fibronectin, collagen, gelatin, hyaluronic acid, polyethylene glycol, functionalized polyethylene glycol, and combinations thereof, iii) whole tissue comprising bone marrow aspirate and peripheral blood, and (iv) cells selected from the group consisting of endothelial cells, hematopoietic cells, and stromal cells, the BMOC device is cultured for a period of about 3 to about 7 days to promote vascularization of the BMOC device and formation of one or both of central marrow and/or periosteal niche microenvironments of bone marrow.

Embodiment 2. The BMOC device of embodiment 1, wherein the at least one central channel further comprises additional cells to promote formation of the bone marrow microenvironments.
Embodiment 3. The multifluidic device of embodiments 1 or 2, wherein the at least two external channels have pores to enable fluid communication with the at least one central channel.
Embodiment 4. The multifluidic device of any of embodiments 1-3, wherein the device further comprises at least one additional external channel proximal to the two external channels.
Embodiment 5. The multifluidic device of any of embodiments 1-4, wherein the at least one additional external channel comprises adhesion promoting materials or additional cells.
Embodiment 6. The multifluidic device of any of embodiments 1-4, wherein the at least one additional external channel comprises a gel.
Embodiment 7. The multifluidic device of any of embodiments 1-6, wherein the gel is either made of adhesion-promoting materials or has adhesion-promoting materials and/or additional cells embedded within it.
Embodiment 8. The multifluidic device of any of embodiments 1-7, wherein the adhesion-promoting materials comprise collagen, polydopamine, fibrin, fibrinogen, fibronectin, polygelatin, ECM proteins or peptides, ECM-like proteins or peptides, and combinations thereof.
Embodiment 9. The multifluidic device of any of embodiments 1-8, wherein the gel comprises fibrin, fibronectin, collagen, gelatin, hyaluronic acid, fibrinogen, thrombin, polyethylene glycol, functionalized polyethylene glycol, and combinations thereof.
Embodiment 10. The multifluidic device of any of embodiments 1-10, wherein the gel is crosslinked by polyethylene glycol.
Embodiment 11. A method of producing a bone-marrow-on-a-chip (BMOC) device, the method comprising:

forming a multichannel microfluidic device from PDMS by soft lithography, wherein the BMOC device comprises at least three channels, at least one media port, and at least one gel port,

wherein the at least three channels comprise at least one central channel and at least two external channels,

wherein one external channel is located on one side of the at least one central channel and proximal to the at least one central channel,

wherein another external channel is located on the opposite side of the at least one central channel and proximal to the at least one central channel;

introducing cells, media and adhesion-promoting materials into the at least one central channel to form an endosteal surface microenvironment,
wherein the cells comprise either (i) differentiated cells selected from the group consisting of osteoblasts and osteoclasts; or (ii) undifferentiated cells selected from the group consisting of MSCs, HSCs, HUVECs, pre-OBs, and other MSC-derived bone-lineage cells,
wherein the media comprises commercially available differentiation media or alphaMEM media optionally supplemented with one or more cytokines, differentiation factors, growth factors, fetal bovine serum, dexamethasone, ascorbic acid, beta-glycerophosphate and/or other osteogenic chemicals, cytokines, or additives,
wherein the adhesion-promoting materials comprise collagen, polydopamine, fibrin, fibrinogen, fibronectin, gelatin, polygelatin, ECM proteins or peptides, ECM-like proteins or peptides, silanes, polyethylene glycol, functionalized polyethylene glycol, and combinations thereof, and
wherein the adhesion-promoting materials form a coating in the at least one central channel;

culturing the BMOC device for about 7 days to about 21 days to promote formation of the endosteal surface;

introducing one or more of pro-vascularization cells or tissue, pro-vascularization media, and a pro-vascularization gel into the one or more central channel(s) of the BMOC device to promote vascularization of the BMOC device,
wherein the pro-vascularization cells or tissue comprise endothelial cells, stromal cells, hematopoietic cells, whole tissue, bone marrow aspirate, and peripheral blood,
wherein the pro-vascularization media comprises endothelial growth media optionally supplemented with one or more cytokines, differentiation factors, and growth factors,
wherein the pro-vascularization gel comprises one or more of fibrin, fibrinogen, fibronectin, collagen, gelatin, hyaluronic acid, polyethylene glycol, functionalized polyethylene glycol, and combinations thereof;

introducing pro-vasculogenesis supporting cells into the outer channel(s),
wherein the outer channel(s) are coated with adhesion-promoting materials comprising one or more of collagen, polydopamine, fibrin, fibrinogen, fibronectin, gelatin, polygelatin, ECM proteins or peptides, ECM-like proteins or peptides, silanes, polyethylene glycol, functionalized polyethylene glycol and combinations thereof, and
wherein the pro-vasculogenesis supporting cells comprise MSCs, fibroblasts, or other pro-vasculogenic primary cells or cell lines; and

culturing the BMOC device in vasculogenic media for about 3 to about 7 days to promote vascularization of the BMOC device and formation of one or both of the central marrow and/or periosteal niche microenvironments of the bone marrow.

Embodiment 12. The method of embodiment 11, wherein the at least one central channel further comprises additional cells to promote formation of the bone marrow microenvironments.
Embodiment 13. The method of embodiments 11 or 12, wherein the at least two external channels have pores to enable fluid communication with the at least one central channel.
Embodiment 14. The method of any of embodiments 11-13, wherein the device further comprises at least one additional external channel proximal to the two external channels.
Embodiment 15. A method of determining the effects of a therapeutic drug on bone marrow comprising:

providing the bone marrow-on-chip (BMOC) device of embodiments 1-10 or generating a BMOC device according to the method of embodiments 11-14;

providing the therapeutic drug to the BMOC device; and

assaying the effects of the therapeutic drug on the bone marrow tissue contained in the BMOC device,

wherein the assayed effects comprise one or more of toxicity, cell differentiation, stem cell migration/mobilization, cytokine expression, and health of the bone marrow tissue or cells, and

wherein the assay comprises one or more of measuring cytokine levels, detecting specific cytokines, microscopy, immunohistochemistry, cell tracking and imaging, vascular network analysis and imaging, cell engraftment analysis, measurement of cell death or killing, and recovery of cells or gels from the BMOC device for single or multi-cell analysis comprising RNA-seq, flow cytometry, Western blotting, ELISA, quantitative PCR, and nucleic acid hybridization and detection.

Embodiment 16. The method of embodiment 15, wherein the therapeutic drug is a small molecule drug, a biologic drug, DNA, RNA, lipids, and/or cells.
Embodiment 17. A method of characterizing a hematopoietic malignancy or other metastatic malignancy comprising:

providing the bone marrow-on-chip (BMOC) device of embodiments 1-10 or generating a BMOC device according to the method of embodiments 11-14, wherein the cells introduced into the at least one channel comprise cells from known malignant cell lines or cells from a sample from a patient having a hematopoietic malignancy; and

assaying the effects of the hematopoietic malignancy or other metastatic malignancy on the bone marrow tissue contained in the BMOC device,

wherein the assayed effects comprise one or more of toxicity, cell differentiation, stem cell migration/mobilization, cytokine expression, and health of the bone marrow tissue or cells, and

wherein the assay comprises one or more of measuring cytokine levels, detecting specific cytokines, microscopy, immunohistochemistry, cell tracking and imaging, vascular network analysis and imaging, cell engraftment analysis, measurement of cell death or killing, and recovery of cells or gels from the BMOC device for single or multi-cell analysis comprising RNA-seq, flow cytometry, Western blotting, ELISA, quantitative PCR, and nucleic acid hybridization and detection.

Embodiment 18. The method of embodiment 17, wherein the hematopoietic malignancy comprises leukemia, lymphoma or myeloma, and wherein the other metastatic malignancies comprise breast cancer, prostate cancer, lung cancer, liver cancer, melanoma, head and neck cancer, gastrointestinal cancer, ovarian cancer, and cervical cancer.
Embodiment 19. The method of embodiment 17 or 18, further comprising the step of introducing a therapeutic to the BMOC device, and assaying the effects of the therapeutic on the bone marrow tissue contained in the BMOC device.
Embodiment 20. The method of any of embodiments 17-19, wherein the therapeutic comprises chemotherapy, radiotherapy, immunotherapy, cell-based therapies, small molecule therapeutic drugs, and biologics.
21. A method of measuring bone marrow stem cell mobilization comprising:

providing the bone marrow-on-chip (BMOC) device of embodiments 1-10 or generating a BMOC device according to the method of embodiments 11-14, wherein the cells introduced into the at least one channel comprise bone marrow stem cells from known cell lines or bone marrow stem cells collected by leukophoresis from a patient; and

assaying the mobility of the bone marrow stem cells contained in the BMOC device,

wherein the assay comprises one or more of measuring cytokine levels, detecting specific cytokines, microscopy, immunohistochemistry, cell tracking and imaging, vascular network analysis and imaging, cell engraftment analysis, measurement of cell death or killing, and recovery of cells or gels from the BMOC device for single or multi-cell analysis comprising RNA-seq, flow cytometry, Western blotting, ELISA, quantitative PCR, and nucleic acid hybridization and detection.

Embodiment 22. The method of embodiment 21, further comprising the step of introducing a therapeutic to the BMOC device, and assaying the effects of the therapeutic on the bone marrow stem cells contained in the BMOC device.
Embodiment 23. The method of embodiments 21 or 22, wherein the therapeutic comprises chemotherapy, radiotherapy, immunotherapy, cell-based therapies, small molecule therapeutic drugs, and biologics.
Embodiment 24. A method of characterizing the effects of radiation exposure on bone marrow comprising:

providing the bone marrow-on-chip (BMOC) device of embodiments 1-10 or generating a BMOC device according to the method of embodiments 11-14, wherein the cells introduced into the at least one channel comprise cells from known cell lines or cells from a sample from a patient; and

assaying the effects of the radiation exposure on the bone marrow tissue contained in the BMOC device,

wherein the assayed effects comprise one or more of toxicity, cell differentiation, stem cell migration/mobilization, cytokine expression, and health of the bone marrow tissue or cells, and

wherein the assay comprises one or more of measuring cytokine levels, detecting specific cytokines, microscopy, immunohistochemistry, cell tracking and imaging, vascular network analysis and imaging, cell engraftment analysis, measurement of cell death or killing, and recovery of cells or gels from the BMOC device for single or multi-cell analysis comprising RNA-seq, flow cytometry, Western blotting, ELISA, quantitative PCR, and nucleic acid hybridization and detection.

Embodiment 25. The method of embodiment 24, wherein the radiation exposure comprises accidental radiation exposure and therapeutic radiation exposure.
Embodiment 26. The method of embodiments 24 or 25, further comprising the step of introducing a countermeasure to the BMOC device to mitigate or prevent the effects of the radiation exposure, and assaying the effects of the countermeasure on the bone marrow tissue contained in the BMOC device.
Embodiment 27. The method of any of embodiments 24-26, wherein the countermeasure comprises cell-based therapies, small molecule therapeutic drugs, and biologics.

While several possible embodiments are disclosed above, embodiments of the present disclosure are not so limited. These exemplary embodiments are not intended to be exhaustive or to unnecessarily limit the scope of the disclosure, but instead were chosen and described in order to explain the principles of the present disclosure so that others skilled in the art may practice the disclosure. Indeed, various modifications of the disclosure in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. The BMOC device of claim 28 further comprising:
first and second external channels;
at least one media port; and
at least one gel port;
wherein the first external channel is located on one side of the central channel and proximal to the central channel;
wherein the second external channel is located on the opposite side of the central channel and proximal to the central channel;
wherein the endosteal surface microenvironment composition comprises a coating or surface treatment comprising:
one or more of polydopamine, fibrin, fibrinogen, fibronectin, collagen, hyaluronic acid, extracellular matrix (ECM) proteins or peptides, ECM-like proteins or peptides, PEG and functionalized PEG; and either
differentiated cells selected from the group consisting of osteoblasts and osteoclasts; or
undifferentiated cells selected from the group consisting of MSCs, HSCs, HUVECs, pre-OBs, and other MSC-derived bone-lineage cells; and
wherein the vascularization composition comprises one or more of:
pro-vascularization media;
a pro-vascularization gel suspension comprising one or more of fibrin, fibrinogen, fibronectin, collagen, gelatin, hyaluronic acid, polyethylene glycol, functionalized polyethylene glycol, and combinations thereof;
whole tissue comprising bone marrow aspirate and peripheral blood; and cells selected from the group consisting of endothelial cells, hematopoietic cells, and stromal cells.

2. The BMOC device of claim 1, wherein the central channel further comprises additional cells to promote formation of the bone marrow microenvironments.

3. The BMOC device of claim 1, wherein the external channels have pores to enable fluid communication with the central channel.

4. The BMOC device of claim 1 further comprising at least one additional external channel proximal to the two external channels.

5. The BMOC device of claim 4, wherein the at least one additional external channel comprises adhesion promoting materials or additional cells.

6. The BMOC device of claim 4, wherein the at least one additional external channel comprises a gel.

7. The BMOC device of claim 6, wherein the gel is either made of adhesion-promoting materials or has adhesion-promoting materials and/or additional cells embedded within it.

8. The BMOC device of claim 7, wherein the adhesion-promoting materials comprise one or more of collagen, polydopamine, fibrin, fibrinogen, fibronectin, polygelatin, ECM proteins or peptides, ECM-like proteins or peptides, and combinations thereof.

9. The BMOC device of claim 6, wherein the gel comprises one or more of fibrin, fibronectin, collagen, gelatin, hyaluronic acid, fibrinogen, thrombin, polyethylene glycol, functionalized polyethylene glycol, and combinations thereof.

10. The BMOC device of claim 9, wherein the gel is crosslinked by polyethylene glycol.

11. A method of comprising:
introducing cells, media and adhesion-promoting materials into a central channel of a multichannel microfluidic device to form an endosteal surface microenvironment;
wherein the cells comprise either (i) differentiated cells selected from the group consisting of osteoblasts and osteoclasts; or (ii) undifferentiated cells selected from the group consisting of MSCs, HSCs, HUVECs, pre-OBs, and other MSC-derived bone-lineage cell;
wherein the media comprises commercially available differentiation media or alphaMEM media optionally supplemented with one or more cytokines, differentiation factors, growth factors, fetal bovine serum, dexamethasone, ascorbic acid, beta-glycerophosphate and/or other osteogenic chemicals, cytokines, or additives;
wherein the adhesion-promoting materials comprise collagen, polydopamine, fibrin, fibrinogen, fibronectin, gelatin, polygelatin, ECM proteins or peptides, ECM-like proteins or peptides, silanes, polyethylene glycol, functionalized polyethylene glycol, and combinations thereof; and
wherein the adhesion-promoting materials form a coating in the central channel;
culturing the multichannel microfluidic device for about 7 days to about 21 days to promote formation of the endosteal surface microenvironment;
introducing one or more of pro-vascularization cells or tissue, pro-vascularization media, and a pro-vascularization gel into the central channel of the multichannel microfluidic device to promote vascularization of the multichannel microfluidic device;
wherein the pro-vascularization cells or tissue comprise endothelial cells, stromal cells, hematopoietic cells, whole tissue, bone marrow aspirate, and peripheral blood;
wherein the pro-vascularization media comprises endothelial growth media optionally supplemented with one or more cytokines, differentiation factors, and growth factors;
wherein the pro-vascularization gel comprises one or more of fibrin, fibrinogen, fibronectin, collagen, gelatin, hyaluronic acid, polyethylene glycol, functionalized polyethylene glycol, and combinations thereof;
introducing pro-vasculogenesis supporting cells into one or more exterior channels of the multichannel microfluidic device, wherein a first external channel is located on one side of the central channel and proximal to the central channel, wherein a second external channel is located on the opposite side of the central channel and proximal to the central channel;
wherein one or more of the exterior channels are coated with adhesion-promoting materials comprising one or more of collagen, polydopamine, fibrin, fibrinogen, fibronectin, gelatin, polygelatin, ECM proteins or peptides, ECM-like proteins or peptides, silanes, polyethylene glycol, functionalized polyethylene glycol and combinations thereof; and
wherein the pro-vasculogenesis supporting cells comprise MSCs, fibroblasts, or other pro-vasculogenic primary cells or cell lines; and
culturing the multichannel microfluidic device in vasculogenic media for about 3 to about 7 days to promote vascularization of the multichannel microfluidic device and formation of one or both of central marrow and/or periosteal niche microenvironments of bone marrow.

12.-14. (canceled)

15. A method comprising:
providing a therapeutic drug to the BMOC device of claim 28; and
assaying effects of the therapeutic drug on bone marrow tissue contained in the BMOC device;
wherein assayed effects include one or more of toxicity, cell differentiation, stem cell migration/mobilization, cytokine expression, and health of the bone marrow tissue or cells; and
wherein the assay comprises one or more of measuring cytokine levels, detecting specific cytokines, microscopy, immunohistochemistry, cell tracking and imaging, vascular network analysis and imaging, cell engraftment analysis, measurement of cell death or killing, and recovery of cells or gels from the BMOC device for single or multi-cell analysis comprising RNA-seq, flow cytometry, Western blotting, ELISA, quantitative PCR, and nucleic acid hybridization and detection.

16. The method of claim 15, wherein the therapeutic drug is selected from the group consisting of a small molecule drug, a biologic drug, DNA, RNA, lipids, cells, and combinations thereof.

17. A method of characterizing a hematopoietic malignancy or other metastatic malignancy comprising:
assaying effects of hematopoietic malignancy or other metastatic malignancy on bone marrow tissue contained in the BMOC device of claim 33;
wherein the cells introduced to the BMOC device comprise cells from known malignant cell lines or cells from a sample from a patient having a hematopoietic malignancy;
wherein assayed effects include one or more of toxicity, cell differentiation, stem cell migration/mobilization, cytokine expression, and health of the bone marrow tissue or cells; and
wherein the assay comprises one or more of measuring cytokine levels, detecting specific cytokines, microscopy, immunohistochemistry, cell tracking and imaging, vascular network analysis and imaging, cell engraftment analysis, measurement of cell death or killing, and recovery of cells or gels from the BMOC device for single or multi-cell analysis comprising RNA-seq, flow cytometry, Western blotting, ELISA, quantitative PCR, and nucleic acid hybridization and detection.

18. The method of claim 17, wherein the hematopoietic malignancy comprises leukemia, lymphoma or myeloma; and
wherein the other metastatic malignancies comprise breast cancer, prostate cancer, lung cancer, liver cancer, melanoma, head and neck cancer, gastrointestinal cancer, ovarian cancer, and cervical cancer.

19. The method of claim 17 further comprising:
introducing a therapeutic to the BMOC device; and
assaying effects of the therapeutic on bone marrow tissue contained in the BMOC device.

20. The method of claim 19, wherein the therapeutic is selected from the group consisting of chemotherapy, radiotherapy, immunotherapy, cell-based therapies, small molecule therapeutic drugs, biologics, and combinations thereof.

21. A method of measuring bone marrow stem cell mobilization comprising:
assaying mobility of bone marrow stem cells contained in the BMOC device of claim 33;
wherein the cells introduced to the BMOC device comprise bone marrow stem cells from known cell lines or bone marrow stem cells collected by leukophoresis from a patient; and
wherein the assay comprises one or more of measuring cytokine levels, detecting specific cytokines, microscopy, immunohistochemistry, cell tracking and imaging, vascular network analysis and imaging, cell engraftment analysis, measurement of cell death or killing, and recovery of cells or gels from the BMOC device for single or multi-cell analysis comprising RNA-seq, flow cytometry, Western blotting, ELISA, quantitative PCR, and nucleic acid hybridization and detection.

22. The method of claim 21 further comprising:
introducing a therapeutic to the BMOC device; and
assaying effects of the therapeutic on bone marrow stem cells contained in the BMOC device.

23. The method of claim 22, wherein the therapeutic is selected from the group consisting of chemotherapy, radiotherapy, immunotherapy, cell-based therapies, small molecule therapeutic drugs, biologics, and combinations thereof.

24. A method of characterizing the effects of radiation exposure on bone marrow comprising:
assaying effects of radiation exposure on bone marrow tissue contained in the BMOC device of claim 33;
wherein the cells introduced to the BMOC device comprise cells from known cell lines or cells from a sample from a patient;
wherein assayed effects include one or more of toxicity, cell differentiation, stem cell migration/mobilization, cytokine expression, and health of the bone marrow tissue or cells; and
wherein the assay comprises one or more of measuring cytokine levels, detecting specific cytokines, microscopy, immunohistochemistry, cell tracking and imaging, vascular network analysis and imaging, cell engraftment analysis, measurement of cell death or killing, and recovery of cells or gels from the BMOC device for single or multi-cell analysis comprising RNA-seq, flow cytometry, Western blotting, ELISA, quantitative PCR, and nucleic acid hybridization and detection.

25. The method of claim 24, wherein the radiation exposure is selected from the group consisting of accidental radiation exposure and therapeutic radiation exposure.

26. The method of claim 24 further comprising:
introducing a countermeasure to the BMOC device to mitigate or prevent the effects of the radiation exposure; and
assaying effects of the countermeasure on bone marrow tissue contained in the BMOC device.

27. The method of claim 26, wherein the countermeasure is selected from the group consisting of cell-based therapies, small molecule therapeutic drugs, biologics, and combinations thereof.

28. A multifluidic bone marrow-on-a-chip (BMOC) device comprising:
a central channel;
an endosteal surface microenvironment composition; and
a vascularization composition;
wherein the endosteal surface microenvironment composition is introduced into the central channel and forms an endosteal surface microenvironment; and
wherein after forming the endosteal surface microenvironment, the vascularization composition is introduced into the central channel and forms of one or both of central marrow and periosteal niche microenvironments of bone marrow;
wherein the endosteal surface microenvironment is formed by culturing the BMOC device for a period of about 7 to about 21 days to promote the formation of the endosteal surface microenvironment; and
wherein the one or both of central marrow and periosteal niche microenvironments of bone marrow is formed by culturing the BMOC device for about 3 to about 7 days to promote vascularization of the BMOC device and the formation of the one or both of the central marrow and the periosteal niche microenvironments of bone marrow.

29. The BMOC device of claim 28, wherein the endosteal surface microenvironment composition comprises cells and adhesion-promoting materials;
wherein the cells comprise either:
differentiated cells selected from the group consisting of osteoblasts and osteoclasts; or
undifferentiated cells selected from the group consisting of Mesenchymal stem cells (MSCs), human stem cells (HSCs), human umbilical vein endothelial cells (HUVECs), pre-osteoblasts (OBs), and other MSC-derived bone-lineage cells; and
wherein the adhesion-promoting materials comprise one or more of collagen, polydopamine, fibrin, fibrinogen, fibronectin, gelatin, polygelatin, extracellular matrix (ECM) proteins or peptides, ECM-like proteins or peptides, silanes, polyethylene glycol, and functionalized polyethylene glycol.

30. The BMOC device of claim 29, wherein the endosteal surface microenvironment composition further comprises media comprising differentiation media, alphaMEM media, and media supplemented with one or more cytokines, differentiation factors, growth factors, fetal bovine serum, dexamethasone, ascorbic acid, beta-glycerophosphate, osteogenic chemicals, and additives.

31. The BMOC device of claim 28, wherein the vascularization composition comprises one or more of pro-vascularization cells or tissue, pro-vascularization media, and a pro-vascularization gel.

32. The BMOC device of claim 31, wherein:
the pro-vascularization cells or tissue comprise endothelial cells, stromal cells, hematopoietic cells, whole tissue, bone marrow aspirate, and peripheral blood;
the pro-vascularization media comprises endothelial growth media optionally supplemented with one or more cytokines, differentiation factors, and growth factors; and
the pro-vascularization gel comprises one or more of fibrin, fibrinogen, fibronectin, collagen, gelatin, hyaluronic acid, polyethylene glycol, functionalized polyethylene glycol, and combinations thereof.

33. A multifluidic bone marrow-on-a-chip (BMOC) device formed by the process of claim 11.
